(19) United States
(12) Patent Application Publication
(10) Pub. No.: US 2022/0285032 A1
(43) Pub. Date: Sep. 8, 2022

(54) Determining Prognosis and Treatment based on Clinical-Pathologic Factors and Continuous Multigene-Expression Profile Scores

(71) Applicant: Castle Biosciences, Inc., Friendswood, TX (US)

(72) Inventors: Kyle R. COVINGTON, Missouri City, TX (US); Bernhard SPIESS, Bellaire, TX (US); Ann QUICK, Houston, TX (US); Sarah KURLEY, Friendswood, TX (US)

(73) Assignee: Castle Biosciences, Inc., Friendswood, TX (US)

(21) Appl. No.: 17/688,215

(22) Filed: Mar. 7, 2022

Related U.S. Application Data
(60) Provisional application No. 63/158,150, filed on Mar. 8, 2021.

(51) Int. Cl. G16H 50/30

(52) U.S. Cl. CPC G16H 50/30

(57) ABSTRACT

Example embodiments relate to determining prognosis and treatment based on clinical-pathologic factors and continuous multigene-expression profile scores. An example embodiment includes a non-transitory, computer-readable medium having instructions stored thereon. The instructions, when executed by a processor, cause the processor to execute a method. The method includes obtaining a plurality of clinical-pathologic factors related to a patient. The clinical-pathologic factors are indicative of risk associated with melanoma. The method also includes obtaining a continuous multigene-expression profile score for the patient. The continuous multigene-expression profile score is based on multiple genes whose expressions are related to melanoma. Further, the method includes determining, based on the plurality of clinical-pathologic factors and the continuous multigene-expression profile score, a risk score for the patient. In addition, the method includes outputting the risk score for use in determining a prognosis and treatment plan.

### CROSS-REFERENCE TO RELATED APPLICATIONS

This application claims priority to U.S. Provisional Application No. 63/158,150, filed Mar. 8, 2021, the disclosure of which is incorporated by reference in its entirety. The contents of U.S. patent application Ser. Nos. 14/193,355; 14/193,378; 15/075,133; 16/745,998; 16/993,401; 61/783,755; and 61/783,788 are herein incorporated by reference in their entirety.

### BACKGROUND

Cancer has a broad impact on present society, both on individual lives and global economies. Many forms of cancer exist, one such form being melanoma. Knowing when a patient is likely to develop melanoma, when the melanoma is likely to metastasize, and/or how likely a patient with melanoma is to survive can help a physician provide guidance to the patient (e.g., provide a prognosis and/or develop a treatment plan).

Further, using baseline clinical and/or pathological factors (i.e., clinical-pathologic factors), a determination can be made as to whether further diagnostic testing is to be performed. However, it is not uncommon for further diagnostic testing to be requested and then, based on the further diagnostic test results, a determination is made that the patient does not have cancer or is at low risk of developing cancer. Such further diagnostic tests can be invasive in some cases, though (e.g., requiring surgery to perform a tissue biopsy). Hence, there can be significant costs whenever an unwarranted diagnostic test is performed.

In assisting in melanoma prognosis, models can be used to determine risks associated with melanoma (e.g., a likelihood of metastasis or a survival rate). These models can, in many cases, be multifactorial. As such, a number of different pieces of data may be collected and/or factored-in when determining such risks associated with melanoma.

### SUMMARY

This disclosure relates to determining prognosis and treatment based on clinical-pathologic factors and continuous multigene-expression profile scores. Some embodiments may include calculating one or more risk scores for a patient based on the both clinical-pathologic factors, as well as continuous multigene-expression profile scores. The risk scores may be determined based on statistical models and/or machine-learned models, for example.

In one aspect, a non-transitory, computer-readable medium is provided. The non-transitory, computer-readable medium has instructions stored thereon. The instructions, when executed by a processor, cause the processor to execute a method. The method includes obtaining a plurality of clinical-pathologic factors related to a patient. The clinical-pathologic factors are indicative of risk associated with melanoma. The method also includes obtaining a continuous multigene-expression profile score for the patient. The continuous multigene-expression profile score is based on multiple genes whose expressions are related to melanoma. In addition, the method includes determining, based on the plurality of clinical-pathologic factors and the continuous multigene-expression profile score, a risk score for the patient. Further, the method includes outputting the risk score for use in determining a prognosis and treatment plan.

In another aspect, a method is provided. The method includes determining a plurality of clinical-pathologic factors related to a patient. The clinical-pathologic factors are indicative of risk associated with melanoma. The method also includes determining a continuous multigene-expression profile score for the patient. The continuous multigene-expression profile score is based on multiple genes whose expressions are related to melanoma. In addition, the method includes providing the plurality of clinical-pathologic factors and the continuous multigene-expression profile score to a computing device. The computing device is configured to calculate, based on the plurality of clinical-pathologic factors and the continuous multigene-expression profile score, a risk score for the patient. The computing device is also configured to output the risk score. Further, the method includes modifying a prognosis or treatment plan based on the risk score.

These as well as other aspects, advantages, and alternatives will become apparent to those of ordinary skill in the art by reading the following detailed description, with reference, where appropriate, to the accompanying drawings.

### BRIEF DESCRIPTION OF THE DRAWINGS

FIG. 1 is an illustration of a computing device, according to example embodiments.

FIG. 2A is an illustration of a method for training a machine-learned model, according to example embodiments.

FIG. 2B is an illustration of a method of making a prediction using a machine-learned model, according to example embodiments.

FIG. 3A is an illustration of a risk score calculation application displayed on a user interface of a computing device, according to example embodiments.

FIG. 3B is an illustration of a risk score calculation application displayed on a user interface of a computing device, according to example embodiments.

FIG. 4 is an illustration of a risk score calculation application displayed on a user interface of a computing device, according to example embodiments.

FIG. 5 is a flowchart illustration of a method, according to example embodiments.

FIG. 6 is a flowchart illustration of a method, according to example embodiments.

FIG. 7 shows the estimated vs. observed risk of 3-year RFS and DMFS in the validation cohort.

FIG. 8 shows 31-GEP improves precision of SLN positivity predictions compared to T-stage based predictions in an independent validation cohort (N=1674) with T1-T4 CM. The integration of the 31-GEP score and clinicopathological features (i31-GEP) is represented by the blue line. Grey shading represents 95% CI. The solid black line represents a perfect match of predicted and observed SLN positive rates. Linear regression shows a y=1.00x+0.01 relationship between predicted and observed positivity demonstrating the close alignment of i31-GEP predicted risk of SLN positivity and observed SLN positivity.

FIG. 9 shows distribution of SLN positivity risk predicted by i31-GEP by T stage. T1a-LR refers to T1a tumors with no high-risk features documented, and T1a-HR refers to patients with a T1a tumor who had risk factors for a positive SLN considered to have a risk between 5-10%. The predicted risk was truncated at 20%. T4a ranged from 9.5-50.0%, and T4b risk ranged from 9.5-58.5%. See supplement for full distribution of predicted SLN positivity, including distribution for T4 tumors

FIG. 10 shows melanoma survival rates in a subset of 312 patients with long-term follow-up stratified by <5% and ≥5% SLN positivity risk by the i31-GEP. The blue line represents the survival of patients with an i31-GEP prediction of SLN positivity <5%, the grey dotted line represents the survival rates of patients with ≥5% positivity that had a negative SLN, and the grey solid line represents the survival rates of patients with ≥5% positivity that had a positive SLN.

FIG. 11 shows a summary of training and validation cohorts.

FIG. 12A-12E show the correlation of individual variables score used in i31-GEP training. Correlation of the continuous 31-GEP score (A), continuous mitotic rate (B), continuous Breslow thickness (C), binary ulceration (D), and continuous age (E) with SLN positivity. Spearman's correlation (rho) and log-likelihood ratios (G2 values) demonstrate a significant correlation between all variables used in training. The GEP continuous score had the highest log-likelihood value, and therefore, had the best fit of all the variables.

FIG. 13 shows the full distribution of SLN positivity risk predicted by i31-GEP by T stage in T1-T4 CM black line is 5%, and the blue line is 10% predicted probability of a positive SLN.

### DETAILED DESCRIPTION

Example methods and systems are contemplated herein. Any example embodiment or feature described herein is not necessarily to be construed as preferred or advantageous over other embodiments or features. The example embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the figures should not be viewed as limiting. It should be understood that other embodiments might include more or less of each element shown in a given figure. Further, some of the illustrated elements may be combined or omitted. Yet further, an example embodiment may include elements that are not illustrated in the figures.

A machine-learned model as described herein may include, but is not limited to: an artificial neural network (e.g., a convolutional neural network, a recurrent neural network, a Bayesian network, a hidden Markov model, a Markov decision process, a logistic regression function, a suitable statistical machine-learning algorithm, and/or a heuristic machine-learning system), a support vector machine, a regression tree, an ensemble of regression trees (also referred to as a regression forest), a decision tree, an ensemble of decision trees (also referred to as a decision forest), or some other machine-learning model architecture or combination of architectures.

“Clinical-pathologic factors,” as used herein, describe any factors pertaining to a patient's health that may provide insight into the likelihood that the patient has a specified disease (e.g., a cancer, such as melanoma). Clinical-pathologic factors may include both signs and symptoms manifested by a patient (e.g., to a physician or clinician during an examination in a clinical setting) and results of laboratory studies (e.g., microscopic review or chemical tests) that examine one or more samples from a patient (e.g., as a result of a tissue biopsy). Example clinical-pathologic factors (e.g., associated with melanoma) include an age of the patient, a gender of the patient, a tumor site location, a histologic type, a Breslow thickness measurement, a transected base measurement, an ulceration measurement, a microsatellites measurement, a mitotic rate, a lymphovascular invasion measurement, a tumor infiltrating lymphocytes measurement, a tumor regression, a sentinel lymph node status, and an in-transit disease/satellites measurement. Other clinical-pathological factors are also possible and are contemplated herein.

A “continuous multigene-expression profile score,” as used herein, describes a score derived for a given disease (e.g., a cancer, such as melanoma) based on a multigene-expression profile. The multigene-expression profile may be correlated with a specific disease. For example, in the example of melanoma, the multigene-expression profile may be based on 31 genes (e.g., 28 prognostic genes and 3 control genes taken from a primary cutaneous melanoma tumor). Other numbers and types of genes in the multigene-expression profile are also possible and are contemplated herein. The multigene-expression profile, itself, may be a result of one or more laboratory tests (e.g., chemical tests) or other tests to determine which of a plurality of genes are expressed within a given patient. Further, some genes within the continuous multigene-expression profile score may have negative correlations with the score. In other words, if the gene is expressed, the score may decrease (e.g., indicating that when that particular gene is expressed, a risk associated with the disease is less than when that particular gene is not expressed). The multigene-expression profile score may be “continuous” in that any number within a range of values is possible for the multigene-expression profile score (e.g., any number between 0 and 1, inclusive). This is different than a “discrete” multigene-expression profile score where only a discrete number of possible scores could be identified (e.g., only scores of 0, 0.25, 0.5, 0.75, or 1; only scores of high-risk, medium-risk, low-risk; etc.). Continuous scores may take into account the degree to which a given gene is expressed, rather than a simple binary determination for each individual gene (e.g., either the gene is expressed or it is not). Hence, continuous multigene-expression profile scores may allow for a higher resolution, and, therefore, higher accuracy when it comes to calculating risk scores and/or ascertaining risk associated with a given disease when compared to discrete multigene-expression profile scores. While “continuous” multigene-expression profile scores are described herein, it is understood that “discrete” multigene-expression profile scores are also contemplated and could also be used. Similarly, while continuous “multigene”-expression profile scores are described herein, it is understood that continuous “single-gene”-expression profile scores are also contemplated and could also be used. Likewise, discrete single-gene-expression profile scores are also contemplated and could also be used.

A “risk score,” as used herein is any indication as to the risk to a patient associated with a given disease (e.g., cancer, such as melanoma). For example, the risk may correspond to the risk that a patient has a given disease, the risk that a patient will develop a given disease, that a patient will suffer a specific event (e.g., death) based on the given disease, the risk that a patient will suffer a specific event (e.g., death) within a certain timeline (e.g., 5 years) based on the given disease, the risk that a patient will contract or develop a related disease, the risk that the disease will present in other bodily regions of the patient (i.e., metastasize), etc. A risk score can represent other risks associated with the disease, as well. The risk score can be represented as a numerical value (e.g., a value between 0 and 1, a percentage between 0 and 100, an integer between 1 and 10, a percentile relative to other patient's in a given class, etc.). The risk score can also be represented by a statement of degree (e.g., high-risk vs. medium-risk vs. low-risk, risk vs. no risk, above-average risk vs. average risk vs. below-average risk, etc.). Example risk scores (e.g., associated with melanoma) include a sentinel lymph node (SLN) metastasis positivity, a recurrence-free survival (RFS) rate, a distance metastasis-free survival (DMFS) rate, and a melanoma specific survival (MSS) rate. Other risk scores are also possible and are contemplated herein.

### I. Overview

Described herein are techniques for generating risk scores for melanoma patients based on clinical-pathologic factors and continuous multigene-expression profile scores. The risk scores may be calculated by a computing device that obtains the clinical-pathologic factors and the continuous multigene-expression profile scores (e.g., from a physician, clinician, or patient) and then generates a risk score based on those pathologic factors. The computing device may then output the risk score (e.g., to a display of the computing device, inserting the risk score in a clinical laboratory report, inserting the risk score in an electronic health record (EHR) associated with the patient, by transmitting the risk score to a user via the Internet, etc.) and/or store the risk score within a memory (e.g., a memory of the computing device or server) for later access. The process of obtaining clinical-pathologic inputs and a continuous multigene-expression profile score, generating a risk score, and outputting the risk score may be implemented in the form of a mobile application (i.e., mobile app) or browser-based application (i.e., browser app or web app), in various embodiments.

In some embodiments, the methods disclosed herein may include one or more physicians (e.g., pathologists or oncologists) and/or clinicians identifying one or more clinical-pathologic factors about a patient. For example, the physician may gather the clinical-pathologic factor(s) by asking a patient questions (e.g., demographic questions), by inspecting (e.g., microscopically) one or more samples gathered from the patient (e.g., as a result of a tissue biopsy), and/or by running tests (e.g., chemical tests) on one or more samples gathered from the patient (e.g., to determine gene expression). Additionally or alternatively, a patient, herself, may provide one or more clinical-pathologic factor(s) to use when calculating risk scores. For example, a patient may input a patient's age, gender, weight, etc. The one or more clinical-pathologic factors may include a variety of factors, such as an age of the patient, a gender of the patient, a tumor site location, a histologic type, a Breslow thickness measurement, a transected base measurement, an ulceration measurement, a microsatellites measurement, a mitotic rate, a lymphovascular invasion measurement, a tumor infiltrating lymphocytes measurement, a tumor regression, a sentinel lymph node status, an in-transit disease/satellites measurement, etc. In some embodiments, such clinical-pathologic factors may be stored in an electronic file associated with the patient (e.g., an electronic health record) maintained by one or more physicians or third-party providers.

Similarly, the continuous multigene-expression profile score may be determined by generating a genetic profile for one or more genes that correspond to the disease (e.g., melanoma) for which the risk score is being calculated. Then, based on the genetic profile, a score may be assigned based on which of the given relevant genes in the profile are expressed. For example, an average may be used (e.g., if a genetic profile assess 5 relevant melanoma genes and only 3 are expressed in the patient, the continuous multigene-expression profile score may be 3 divided by 5, or 0.6). Alternatively, a weighted average may be used to determine the continuous multigene-expression profile score (e.g., in order to value the expression or non-expression of certain genes within the profile over others). As indicted in these examples, the continuous multigene-expression profile score may take on any value between 0 and 1, inclusive (e.g., depending on the number of genes expressed out of the total number of relevant genes). Other ways of generating a continuous multigene-expression profile score are also possible and are contemplated herein.

The continuous multigene-expression profile score may be a continuous score (e.g., be capable of taking on any real number between 0 and 1). This may be an improvement over other techniques where the gene expression scores are only expressed in discrete increments (e.g., gene expression scores that only have two possible values, four possible values, eight possible values, etc.) because a continuous value may be more representative of the patient's condition and, ultimately, usable to generate a risk score with greater accuracy.

One or more of the clinical-pathologic factors and the continuous multigene-expression profile score may then be obtained by a computing device. The computing device may take different forms in various embodiments. For example, the computing device may include a mobile device (e.g., a mobile phone using a mobile app), a tablet computing device (e.g., using a mobile app), a personal computer (e.g., using a browser-based app that includes a web interface or an installed application), a server, etc. Other computing devices are also possible. Further, the computing device may include a processor and a non-transitory, computer-readable medium having instructions stored thereon. The instructions may be executable by the processor to perform one or more of the methods described herein. The non-transitory, computer-readable medium may correspond to one or more portions of non-volatile memory (e.g., a read-only memory (ROM), such as a hard drive) of the computing device, for example. Additionally, the computing device may include one or more volatile memories (e.g., a random-access memory (RAM)) used by the processor in the course of performing one or more of the methods described herein while executing the instructions.

In some embodiments, obtaining the clinical-pathologic factors and the continuous multigene-expression profile score may include one or more physicians/clinicians (e.g., one or more physicians/clinicians who initially measured the respective clinical-pathologic factors and/or generated the continuous multigene-expression profile score) inputting the clinical-pathologic factors and/or the continuous multigene-expression profile score into the computing device (e.g., using an input device such as a keyboard, computer mouse, microphone, etc. of the computing device). Additionally or alternatively, the computing device may receive one or more of the clinical-pathologic factors or the continuous multigene-expression profile score from a different computing device. For example, when the computing device obtaining the clinical-pathologic factors and the continuous multigene-expression profile score is a server, an additional computing device (e.g., a mobile device) may receive inputs (e.g., via a mobile app) from a user (e.g., a physician) indicative of the clinical-pathologic factor(s) and/or the continuous multigene-expression profile score and then the clinical-pathologic factor(s) and/or the continuous multigene-expression profile score may be transmitted to the server via the public Internet or over a local network (e.g., a local IEEE 802.11 standards (WIFI) network).

In other embodiments, a user (e.g., a first physician) may input clinical-pathologic factor(s) and/or the continuous multigene-expression profile score into a first computing device (e.g., a tablet computing device using a browser-based app) and the clinical-pathologic factor(s) and/or the continuous multigene-expression profile score may then be transmitted to a different computing device (e.g., a mobile device of a second physician) for analysis/computation.

Still further, obtaining the clinical-pathologic factors and/or the continuous multigene-expression profile score may include the computing device retrieving the clinical-pathologic factors and/or the continuous multigene-expression profile score from one or more storage locations (e.g., from a memory associated with a server that stores information related to the patient). In some embodiments, clinical-pathologic factors and/or the continuous multigene-expression profile score may be obtained by the computing device from multiple sources. For example, the computing device may receive a first set of clinical-pathologic factors from a mobile device of the patient, a second set of clinical-pathologic factors from a tablet computing device of a physician, and the continuous multigene-expression profile score from a server (e.g., associated with an electronic health record of the patient).

Additionally or alternatively, the computing device obtaining the clinical-pathologic factors or the continuous multigene-expression profile score may include the computing device receiving raw data and then analyzing that data to arrive at the clinical-pathologic factors or the continuous multigene-expression profile score. For example, the computing device may receive a continuous multigene-expression profile and then calculate the continuous multigene-expression profile score using an average or weighted average (e.g., as described above). Other techniques for obtaining the clinical-pathologic factors and/or the continuous multigene-expression profile score are also possible and are contemplated herein.

In some embodiments, prior to a user providing the clinical-pathologic factor(s) and/or the continuous multigene-expression profile score to the computing device, the user may need to provide user login credentials (e.g., a username, a password, a personal identification number (PIN), a generated code, etc.). The computing device may validate such user login credentials against previously authenticated login credentials associated with authenticated users. For example, the computing device may ensure that a supplied username and password combination match a previously authenticated/stored username and password combination within a repository associated with the computing device (e.g., within a memory of the computing device or a cloud storage associated with the computing device). The user login credentials may also be used by the computing device to identify a type of user accessing the computing device (e.g., as a physician, a clinician, an insurer, a patient, etc.). Further, there may be certain permissions associated with the type of user accessing the computing device (e.g., a physician is permitted to view/edit all information for all of that physician's patients whereas a patient is only permitted to view all the information associated with that patient or a select subset of the information associated with that patient). Still further, the user login credentials may associate certain users with other users. For example, a user login credential representing a physician may have associations with other users representing patients of that physician. In this way, the physician's user login credentials may be usable to view/edit the pathologic factors and/or generated risk scores associated with that physician's patients (e.g., and no other patients). Such protocols may be usable to ensure compliance with governmental privacy regulations (e.g., regulations associated with the Health Insurance Portability and Accountability Act (HIPAA)).

Upon the computing device receiving the associated pathologic factors, the computing device may then calculate one or more risk scores associated with the patient based on the clinical-pathologic factors and the continuous multigene-expression risk score. The risk scores may represent different probabilities associated with the patient's melanoma condition. For example, the risk scores may include a SLN metastasis positivity, a RFS rate, a DMFS rate, and/or a MSS rate. Because these risk score(s) correspond to rates/probabilities, the risk score(s) may have values between 0 and 1. Additionally or alternatively, though, the risk score(s) may have other values. For example, the risk score(s) may be scaled to have a value between 0 and 100. Additionally, the risk score(s) may be scaled relative to other patient's having similar age, gender, etc. as the present patient and the risk score(s) may be displayed as a percentile relative to other patient's having similar characteristics. Each of the risk score(s) may be calculated differently and/or have a different range of possible values.

Further, the risk scores may be calculated by the computing device according to one or more models/equations based on the clinical-pathologic factors and/or continuous multigene-expression profile scores. Such models/equations may be determined by studying populations of previous melanoma (or other cancer or disease under study) patients and their outcomes. For example, a machine-learned model (e.g., an artificial neural network (ANN)) may be trained using previous melanoma patient data as labeled training data. The machine-learned model may be stored in the non-transitory, computer-readable medium of the computing device, for example. In some embodiments, the clinical-pathologic factors and the continuous multigene-expression profile score of the current patient may be fed into the machine-learned model and the machine-learned model may determine the one or more risk scores based on the clinical-pathologic factors and the continuous multigene-expression profile score. Additionally or alternatively, the computing device may determine the risk score(s) by applying a statistical analysis (e.g., a Cox regression analysis) using each of the clinical-pathologic factors and continuous multigene-expression profile score. In some embodiments, determining the risk score(s) may include using the clinical-pathologic factors and/or the continuous multigene-expression profile score as variables in an equation that has associated coefficients and/or exponentials. For example, each of the different types of risk score(s) may be represented by one or more polynomials.

If one or more of the clinical-pathologic factors and/or the continuous multigene-expression profile score used in determining a given risk score (e.g., a MSS rate) is unavailable (e.g., was not supplied by the physician or retrieved from the patient's electronic health record), a default value may be inserted (e.g., the mean value or the median value across all patients) to permit the calculation to be performed. In other embodiments, the given risk score may be calculated with the missing clinical-pathologic factor(s) or continuous multigene-expression profile score set to a value corresponding to the maximum or minimum values. Additionally or alternatively, if not all clinical-pathologic factors and/or the continuous multigene-expression profile score used to determine a given risk score are present, that given risk score may not be calculated and/or may be calculated but flagged as being potentially inaccurate/unreliable. In still other embodiments, a range of values for a given risk score may be calculated by inserting all possible values for the unsupplied clinical-pathologic factor(s) or continuous multigene-expression profile score into the calculation and generating a corresponding set of risk scores based on those possible values. Further, the computing device may output (e.g., may display to a user or transmit a communication, such as an email or a text, to a user) a request for the unsupplied clinical-pathologic factor(s) or continuous multigene-expression profile score in order to perform and/or revise the associated risk score calculation.

After the risk score(s) associated with the patient are calculated, they may be provided by the computing device. Providing the risk score(s) may include displaying the risk score(s) on a display (e.g., a light-emitting diode (LED) display or a liquid-crystal display (LCD)) of the computing device (e.g., to the physician or the patient using the computing device). In embodiments where the computing device is a mobile device (e.g., executing a mobile application), the risk score(s) may be displayed as a pop-notification, for example. Further, providing the risk score(s) may include transmitting the risk score(s) to one or more other computing devices (e.g., over the public Internet). For example, providing the risk score(s) may include texting, emailing, and/or otherwise communicating the risk score(s) to the patient and/or the patient's physician. Further, providing the risk score(s) may include storing the risk scores in one or more memories (e.g., a server associated with an EHR of the patient) for later access. For example, the risk score(s) may be associated with the login credentials of a patient and/or a patient's physician and stored within a memory for later access (e.g., solely by the patient and/or patient's physician).

Using the risk score(s) (e.g., once they are provided by the computing device based on the pathologic factors), the patient's physician may provide the patient with an updated prognosis. Additionally or alternatively, the computing device may, itself, provide a prognosis to the patient directly (e.g., when the patient input the pathologic factors herself). Further, a patient's physician may generate or revise a treatment plan for the patient based on the risk score(s) provided by the computing device.

Additionally, after the risk score(s) are provided, it may be determined that additional clinical-pathologic testing is to be performed and/or that a continuous multigene-expression profile should be generated/scored. For example, if all the clinical-pathologic factors needed to fully calculate a given risk score were not present at the time of calculation (e.g., if a default value was used for one of the pathologic factors in the calculation or a range of risk score values were calculated), it may be desirable to perform a clinical-pathologic test to determine an additional clinical-pathologic factor that may be fed into the calculation. Hence, in some embodiments, after providing the risk score(s), the computing device may receive one or more additional clinical-pathologic factors, one or more revised clinical-pathologic factors (i.e., a different value for a clinical-pathologic factor that was previously obtained by the computing device), a continuous multigene-expression profile score, and/or a revised continuous multigene-expression profile score (i.e., a different continuous multigene-expression profile score than was previously obtained by the computing device). For example, after calculating and providing a set of risk score(s), the computing device may obtain a continuous multigene-expression profile related to melanoma (e.g., based on a gene-expression study that was completed after the risk score(s) were first calculated by the computing device). Upon receiving the one or more additional and/or revised clinical-pathologic factors or continuous multigene-expression profile score, revised risk score(s) may be calculated and the revised risk score(s) may then be provided. This process of receiving additional and/or revised pathologic factor(s) and then calculating revised risk score(s) may be performed multiple times. In some embodiments, the risk score(s) from each iteration may be stored (e.g., in a non-volatile memory of the computing device) and used to generate a plot of risk score(s) over time.

As described above, it is not uncommon, using traditional diagnostic techniques, for further diagnostic testing to be requested and then, based on the further diagnostic test results, a determination is made that the patient does not have cancer or is at low risk of developing cancer. Such further diagnostic tests can be invasive in some cases, though (e.g., requiring surgery to perform a tissue biopsy). Hence, there can be significant costs whenever an unwarranted diagnostic test is performed.

The techniques described herein provide improvements to diagnosing diseases (e.g., cancer, such as melanoma) by increasing the accuracy of the preliminary diagnosis and, thereby, reducing the rate at which unnecessary additional (potentially invasive) diagnostic tests are to be performed. One way in which the techniques described herein provide such improvements is by combining clinical-pathologic factors and continuous multigene-expression profile scores to determine a risk score (e.g., as opposed to analyzing only clinical-pathologic factors or only continuous multigene-expression profile scores). As just one example of such an improvement, the improved diagnostic accuracy of the techniques described herein is evaluated below with respect to melanoma.

The American Joint Committee on Cancer (AJCC) maintains a tumor characteristics, nodal disease burden, and tumor metastasis (TNM) staging system to estimate each patient's risk of death due to melanoma. Further, detection of melanoma metastasis to the lymph node may qualify a patient specific types of treatments for melanoma (e.g., adjuvant therapy). Unfortunately, many patients (e.g., as many as 88%) who undergo a sentinel lymph node biopsy (SLNB) may have a negative result and therefore be exposed to surgical risks unnecessarily.

The National Comprehensive Cancer Network (NCCN) recommends that: (1) clinicians offer SLNB to patients if they have a risk of positive SLN greater than 10% (for T2-T4 tumors), (2) clinicians discuss the possibility of SLNB if the risk is between 5% and 10% (for T1a tumors with high-risk features or a T1b tumor), and (3) clinicians do not recommend an SLNB if the risk is less than 5% (T1a tumors without high-risk features). Based on these guidelines (and others like it), as well as a desire to not expose patients to the unnecessary risk of surgery, it is important to properly place a patient in these (or similar) categories. Further, especially for patients in the 5%-10% risk range, since a judgement call is to be made by the physician/patient, it is important to accurately determine the exact risk within a given category.

Some methods of making SLN positivity predictions include performing logistic regression and applying a point system to determine risk. Such techniques did not integrate features of tumor biology. Further, such techniques have traditionally been rather rigid as to what clinical-pathologic features they analyze in determining a risk. Additionally, such traditional techniques have not explored integrated a continuous multigene-expression profile score along with clinical-pathologic factors to determine a risk score.

The techniques described herein were validated in an independent cohort of N=1674 patients with T1-T4 tumors. The techniques herein predicted that 27.7% (464/1674) of patients had a predicted probability of <5%, and 41.6% (696/1674) had a predicted probability of >10% compared with just 8.5% with <5% SLN positivity risk for a low-risk T1a designation. In the validation cohort, 377 tumors were designated as T1a (235 of which had one or more high-risk features), and 328 as T1b. The hybrid clinical-pathological/continuous multigene-expression profile score techniques herein re-classified (when compared to standard techniques) 68.5% (161/235) of T1a tumors with at least one high-risk feature, and 40.9% (134/328) of T1b as low risk (<5% risk of SLN positivity) for a total of 52.4% of higher-risk T1 tumors re-classified as <5% risk. Moreover, the techniques herein re-classified (when compared to standard techniques) 4.7% (11/235) of patients with a T1a tumor and at least one high-risk feature and 14.3% (47/328) T1b tumors as having >10%, risk re-classifying a total of 10.3% of higher-risk T1 tumors as having a predicted risk >10%.

To summarize, of the 563/1542 patients with SLN positivity risk classified by T-stage as between 5-10%, the hybrid techniques described herein re-classified 62.7% (353/563) to <5% or >10% SLN positivity risk. This would correspond to a much easier decision by the physician on behalf of the 353 patients that were re-classified (i.e., under the guidelines above, they should decisively recommend or decisively reject SLNB tests for those patients, rather than in the previous indecisive middle category). Similarly, validation of cases in the T2 population demonstrated that 12.5% (52/416) of T2a tumors and 4.2% (5/118) of patients with T2b tumors were predicted to have a <5% risk and 44.7% (186/416) of T2a and 44.1% (52/118) of T2b cases had a 5-10% risk of SLN positivity, providing potentially meaningful risk reduction within T2 tumors while identifying more precise risk for those T2 cases with a >10% risk of SLN positivity.

On the other hand, while only 0.3% (1/303) of T3 cases had a <5% risk prediction, 10.2% (31/303) of cases had a risk between 5-10% with the majority of T3 cases having a risk >10% as expected. Validation in patients with T4 tumors confirmed that while the majority (96%) had SLN positivity predictions higher than 10%, the range was wide (9.5-58%), which may be important in SLNB discussions for patients with comorbidities in which the benefit/risk ratio of SLNB is concerning. Overall validation demonstrated that the techniques described herein improved precision of risk predictions over T stage alone.

As indicated above, risk determination for melanoma patients was improved by using the hybrid clinical-pathologic factors/continuous multigene-expression profile score technique described herein rather than previously used techniques. As a result, the techniques described herein would reduce the unnecessary number of required invasion surgeries and enhance physician confidence when providing diagnostic and treatment recommendations to patients.

While the above-described improvements were demonstrated in melanoma patients, it is understood that similar improvements may result by applying the techniques described herein to other cancers or other diseases entirely.

### II. Example Systems

The following description and accompanying drawings will elucidate features of various example embodiments. The embodiments provided are by way of example, and are not intended to be limiting. As such, the dimensions of the drawings are not necessarily to scale.

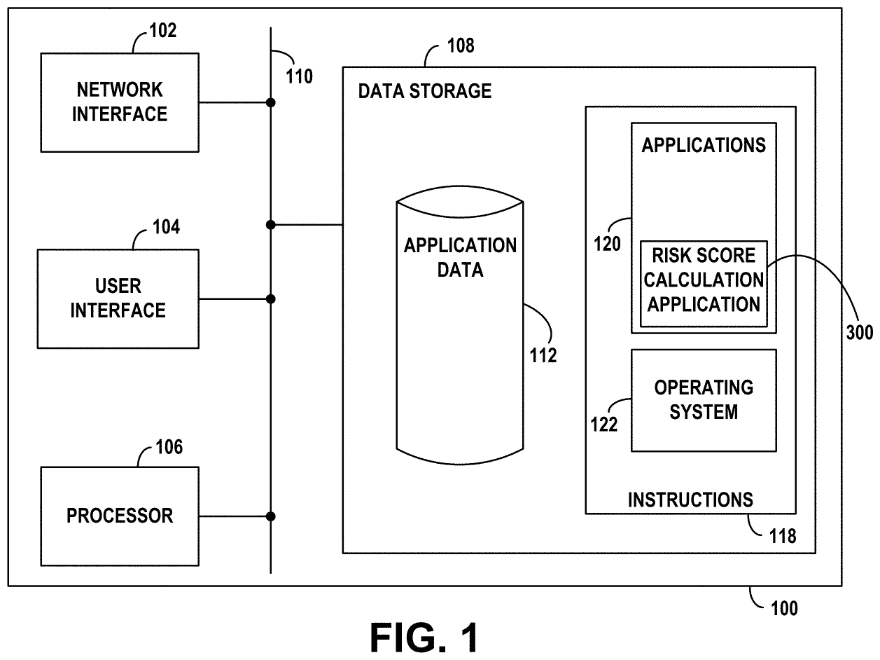

FIG. 1 is a simplified block diagram showing some of the components of an example computing device 100. The computing device 100 may correspond to a computing device configured to perform the functions described throughout this disclosure (e.g., in communication with one or more other computing devices using a web browser and/or an application). In various embodiments, the computing device 100 may be a mobile computing device (e.g., a smartphone), a desktop computing device, a laptop computing device, a tablet computing device, or a wearable computing device (e.g., a smartwatch or a smart wristband). As illustrated in FIG. 1, the computing device 100 may include a network interface 102, a user interface 104, a processor 106, and data storage 108. The network interface 102, the user interface 104, the processor 106, and/or the data storage 108 may be communicatively linked together by a bus 110 (e.g., an electrical interconnect defined on one or more printed circuit boards).

The network interface 102 may be used by the computing device 100 to communicate with other computing devices over one or more networks (e.g., the public Internet). In some embodiments, the network interface 102 may include a wired interface (e.g., Ethernet). Additionally or alternatively, the network interface 102 may include a wireless interface, such as WIFI). Other interfaces may be included in the network interface 102 and are contemplated herein.

The user interface 104 may function to allow computing device 100 to receive input from and/or provide output to a user. As such, the user interface 104 may include inputs (e.g., a keypad, a keyboard, a touch-screen, a computer mouse, a microphone, a microphone jack, etc.) and/or outputs (e.g., a cathode-ray tube (CRT) display, a LCD, a LED display, a speaker, a speaker jack, headphones, a headphone jack, etc.).

The processor 106 may include one or more general purpose processors (e.g., microprocessors) and/or one or more special-purpose processors (e.g., graphics processing units (GPUs) or application-specific integrated circuits (ASICs)). In some embodiments, for example, the processor 106 may include special-purpose processors capable of generating a machine-learned model and/or using a machine-learned model to perform analyses as described herein.

The data storage 108 may include one or more volatile and/or non-volatile memories. For example, the data storage may include a RAM, a ROM, a hard drive, a solid state drive, etc. In some embodiments, the data storage 108 may be partially or wholly integrated with the processor 106 (e.g., a level 1 (L1) cache or a level 2 (L2) cache within a central processing unit). The data storage 108 may include removable components (e.g., a flash drive) and/or non-removable components (e.g., a hard disk attached to a motherboard).

The processor 106 may be configured to execute instructions 118 (e.g., compiled or non-compiled program logic and/or machine code) stored in the data storage 108 to carry out the methods described herein. Hence, the data storage 108 may include a non-transitory computer-readable medium, having stored thereon program instructions that, when executed by the processor 106, cause the processor 106 to carry out any of the methods, processes, or operations disclosed in this specification and/or the accompanying drawings. In some embodiments, the processor 106 may use the application data 112 while executing the instructions 118.

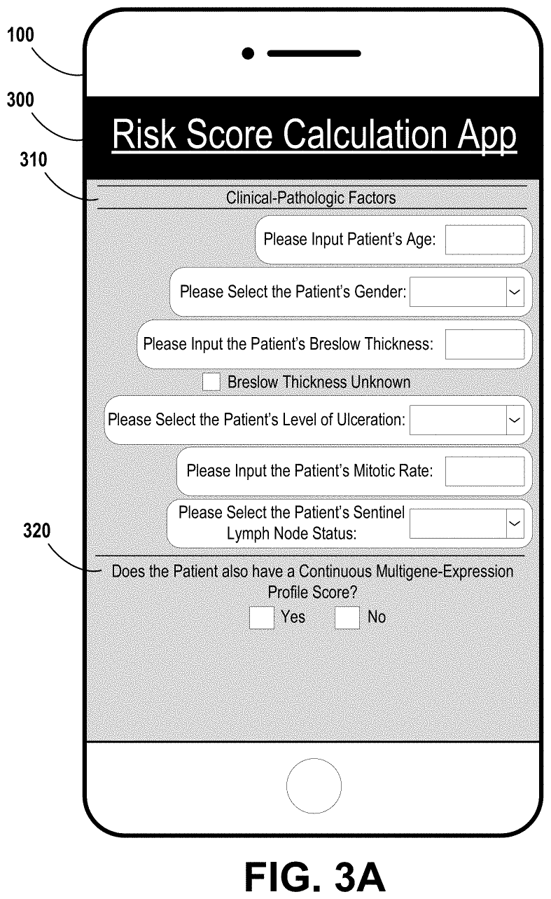

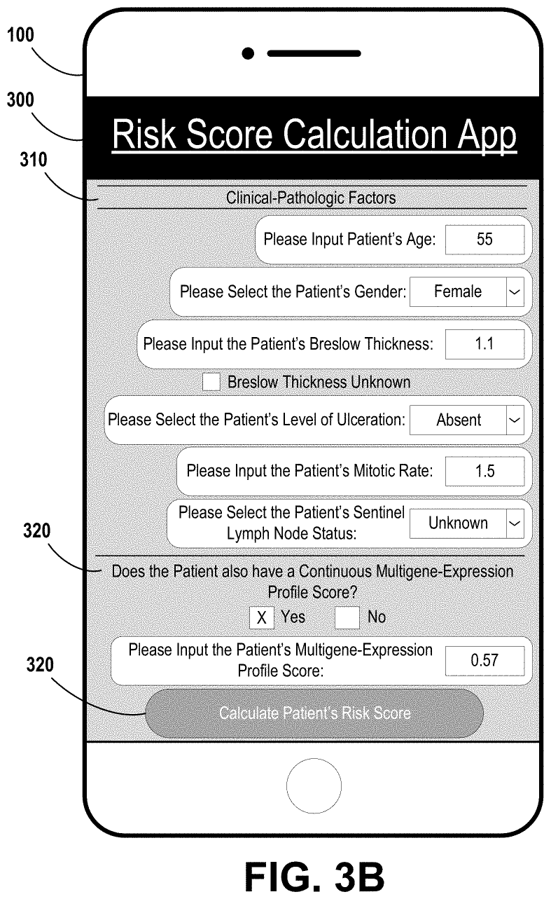

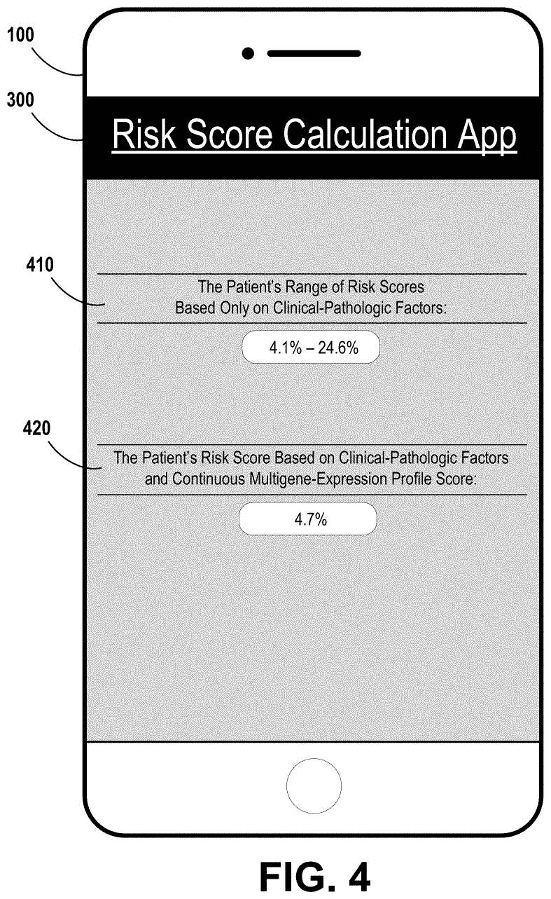

In some embodiments, the instructions 118 may include an operating system 122 (e.g., an operating system kernel, device driver(s), and/or other modules) and one or more applications 120 (e.g., mobile applications, sometimes referred to as “apps”). For example, the applications 120 may include an email app, a web browser, a social networking app, and/or a dedicated app to perform the functions/calculations described herein (e.g., a risk score calculation application 300 as shown and described with reference to FIGS. 3A-4). As described above, the processor 106 may access the application data 112 when executing the applications 120.

The applications 120 may communicate with the operating system 122 through one or more application programming interfaces (APIs). These APIs may facilitate, for instance, the applications 120 reading and/or writing the application data 112, transmitting or receiving information via the network interface 102, receiving and/or displaying information on the user interface 104, etc.

Additionally, the applications 120 may be downloadable to the computing device 100 through one or more online application stores or application markets (e.g., using the network interface 102). However, application programs can also be installed on the computing device 100 in other ways, such as via a web browser or through a physical interface (e.g., a universal serial bus (USB) port) on the computing device 100.

While many of the techniques and functions described herein may be performed by the processor 106 executing one of the applications 120 that is dedicated to determining risk scores for patients (e.g., a risk score calculation app), it understood that other ways for the computing device 100 to perform such techniques and functions are also possible and are contemplated herein. For example, the processor 106 may execute a web browser app of the applications 120 to communicate with one or more other computing devices using the network interface 102. In such a case, some or all of the calculations may be performed remotely (e.g., on a server computing device). Such an embodiment may be referred to as a “browser-based app” where the computing device 100 provides data (e.g., application data 112) to a different computing device for analysis. Such an interaction between the computing device 100 and another computing device may be performed using an API or a browser-based language (e.g., JavaScript).

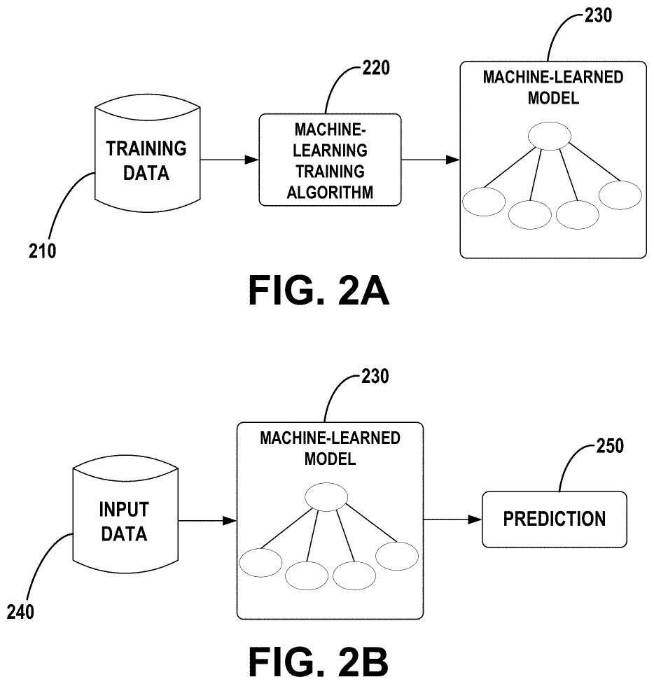

FIG. 2A illustrates a method of training a machine-learned model 230 (e.g., an artificial neural network), according to example embodiments. The method of FIG. 2A may be performed by a computing device (e.g., the computing device 100 illustrated in FIG. 1), in some embodiments. As illustrated, the machine-learned model 230 may be trained using a machine-learning training algorithm 220 based on training data 210 (e.g., based on patterns within the training data 210). While only one machine-learned model 230 is illustrated in FIGS. 2A and 2B, it is understood that multiple machine-learned models could be trained simultaneously and/or sequentially and used to perform the predictions described herein. Ultimately, a prediction 250 may be made using the trained machine-learned model 230. For example, the machine-learned model 230 may be used to determine a risk score for a patient based on input data 240 (i.e., patient clinical-pathologic factors and/or a continuous multigene-expression profile score) within the risk score calculation application 300 described below with reference to FIGS. 3A-4

The machine-learned model 230 may include, but is not limited to: an artificial neural network (e.g., a convolutional neural network, a recurrent neural network, a Bayesian network, a hidden Markov model, a Markov decision process, a logistic regression function, a suitable statistical machine-learning algorithm, and/or a heuristic machine-learning system), a support vector machine, a regression tree, an ensemble of regression trees (also referred to as a regression forest), a decision tree, an ensemble of decision trees (also referred to as a decision forest), or some other machine-learning model architecture or combination of architectures. The machine-learning training algorithm 220 may involve supervised learning, semi-supervised learning, reinforcement learning, and/or unsupervised learning. Similarly, the training data 210 may include labeled training data and/or unlabeled training data.

The training data 210 may include clinical-pathologic data and/or continuous multigene-expression profile scores coupled with outcomes for previously observed patients. For example, the training data 210 may include data for 1,000 patients. For each of the 1,000 patients, the training data 210 may include clinical-pathologic data (e.g., for a range of clinical-pathologic factors), the continuous multigene-expression profile score (e.g., for a variety of genes), and the outcome for the patient (e.g., whether the patient survived for a certain length of time). Using the clinical-pathologic factors and the continuous multigene-expression profile score (e.g., the expression, or lack thereof, for each gene within the profile), the machine-learning training algorithm 220 may attempt to make a prediction about the outcome of a patient. If the predicted outcome for that given patient matches the actual outcome for a patient within the training data 210, this may reinforce the machine-learned model 230 being developed by the machine-learning training algorithm 220. If the predicted outcome for that give patient does not match the actual outcome for a patient within the training data 210, the machine-learned model 230 being developed by the machine-learning training algorithm 220 may be modified to accommodate the difference (e.g., the weight of a given factor within the artificial neural network of the machine-learned model 230 may be adjusted). Additionally or alternatively, in some embodiments, the machine-learning training algorithm 220 may enforce additional rules during the training of the machine-learned model 230 (e.g., by setting and/or adjusting one or more hyperparameters).

Once the machine-learned model 230 is trained by the machine-learning training algorithm 220 (e.g., using the method of FIG. 2A), the machine-learned model 230 may be used to make one or more predictions. For example, a computing device (e.g., the computing device 100 illustrated in FIG. 1), may make a prediction 250 using the machine-learned model 230 based on input data 240, as illustrated in FIG. 2B.

As illustrated in FIG. 2B, the machine-learned model 230 can receive input data 240 and generate and output one or more predictions 250 about input data 240. For example, in some embodiments described herein, the input data 240 may include one or more clinical-pathologic factors and/or a continuous multigene-expression profile score for a patient. The machine-learned model 230 (e.g., an artificial neural network) may take this patient data and produce the prediction 250 based on this input data 240. The prediction 250 may include a risk score or a range of risk scores. For example, when all possible clinical-pathologic factors and the complete continuous multigene-expression profile score for the patient have been provided in the input data 240, the machine-learned model 230 may generate a single risk score (e.g., based on weights for each factor provided in the input data 240 using an artificial neural network of the machine-learned model 230). However, if some subset of the possible clinical-pathologic factors and/or one or more genes within the continuous multigene-expression score for a patient have not been provided within the input data 240, the machine-learned model 230 may provide a range of risk scores (e.g., the range of risk scores corresponding to all possible combinations of values of the unknown factors for the patient). Alternatively, when some subset of the possible clinical-pathologic factors and/or one or more genes within the continuous multigene-expression score for a patient have not been provided within the input data 240, the machine-learned model 230 may still calculate a single risk score by applying an average population values to the unknown factors or by applying values to the unknown factors based on the values of the known factors (e.g., such inferences may be made by the machine-learned model 230 based on information contained within the training data 210 and incorporated into the machine-learned model 230 by the machine-learning training algorithm 220).

While the same computing device (e.g., the computing device 100 of FIG. 1) may be used to both train the machine-learned model 230 (e.g., as illustrated in FIG. 2A) and make use of the machine-learned model 230 to make a prediction 250 (e.g., as illustrated in FIG. 2B), it is understood that this need not be the case. In some embodiments, for example, a computing device may execute the machine-learning training algorithm 220 to train the machine-learned model 230 and may then transmit the machine-learned model 230 to another computing device for use in making one or more predictions 250. In this context of this disclosure, for example, a computing device may be used to initially train the machine-learned model 230 and then this machine-learned model 230 could be stored for later use. For example, the machine-learned model 230 could be trained and then packaged with a risk score calculation application 300 and distributed to one or more computing devices (e.g., a mobile computing device, such as the computing device 100 of FIG. 1) as a part of the risk score calculation application 300. Then, the computing device 100 that receives the risk score calculation application 300 may simply make use of the machine-learned model 230 without having to train it.

FIG. 3A-4 illustrate the computing device 100 as a mobile computing device (e.g., smartphone). Such a mobile computing device may include one or more processors (e.g., the processor 106 illustrated in FIG. 1) configured to execute a set of instructions stored within a non-transitory, computer-readable medium (e.g., the instructions 118 within the data storage 108, as illustrated in FIG. 1). The set of instructions may correspond to one of the application 120 (e.g., a risk score calculation application 300). The following figures are used to show and describe potential features of the risk score calculation application 300. Executing the risk score calculation application 300 may include the processor 106 accessing one or more pieces of application data 112 from the data storage 108 that are associated with the risk score calculation application 300. For example, the processor 106 may retrieve and use ranges of values for different clinical-pathologic factors, ranges of values for continuous multigene-expression profile scores, data related to a specific patient, images related to a specific patient, a patient's electronic health record, etc.

It is understood that the processes of the risk score calculation application 300 may be equally performed by other forms of the computing device 100. For example, the computing device 100 may additionally or alternatively include a tablet computing device, a wearable computing device (e.g., APPLE WATCH), a laptop computing device, or a desktop computing device. Further, it is understood that the processes of the risk score calculation application 300 may equally be carried out partially on a computing device in communication with the computing device 100. This may be the case if the risk score calculation application 300 corresponds to a browser-based application, for example.

In order to carry out various functions of the risk score calculation application 300, the computing device 100 may communicate with one or more servers. For example, the computing device 100 may communicate with one or more public cloud servers that are running using MICROSOFT AZURE or AMAZON WEB SERVICES. Communication with cloud servers may occur via a network, such as the public Internet, using the network interface 102. While various functions and features may be shown and described herein as being carried out by/on the computing device 100, it is understood that any individual feature may equally be executed on the one or more servers. For example, as herein shown and described, the computing device 100 may determine a risk score based on a given set of clinical-pathologic factors and a continuous multigene-expression profile score associated with a patient (e.g., to determine a cancer risk posed to the patient). It is understood that, instead, the patient data (e.g., the patient's clinical-pathologic information and the patient's continuous multigene-expression profile score) could be transmitted to one or more servers, and the one or more servers could perform the same image calculation. The server(s) may then provide the results to the computing device 100, which may then output the risk score (e.g., by displaying the risk score on a display of the user interface 104 or inserting the risk score into a patient report). Interactions between the computing device 100 and the server(s) may occur based on an API associated with the risk score calculation application 300, in some embodiments. For example, API commands may be used to transmit information from the computing device 100 to the server(s) and/or to instruct the servers to perform certain calculations.

Similarly, while some data (e.g., clinical-pathologic information about a patient) may be described as being stored locally on the computing device 100 (e.g., as application data 112 within the data storage 108) or input into the computing device 100 using the user interface 104, it is understood that such data could additionally or alternatively be stored within a server. This data may be accessible by the computing device 100 when requesting that the one or more servers perform one or more tasks. Additionally or alternatively, the servers may act merely as a data repository, and the computing device 100 may retrieve data (e.g., patient clinical-pathologic information) from the one or more servers, yet still perform the risk score calculations within the risk score calculation application 300 locally on the computing device 100.

FIGS. 3A-4 are illustrations of a risk score calculation application (e.g., the risk score calculation application 300 illustrated in FIG. 1), according to example embodiments. For example, FIGS. 3A-4 may illustrate the display of a user interface (e.g., user interface 104 of FIG. 1) of a computing device (e.g., the computing device 100 of FIG. 1) as a processor (e.g., the processor 106 of FIG. 1) executes the risk score calculation application 300. The risk score calculation application 300 may be a mobile application, as illustrated in FIGS. 3A-4. Alternatively, the risk score calculation application 300 may correspond to a browser-based application (i.e., a web application). In such embodiments, the risk score calculation application 300 may be executed within a web browser (e.g., on a mobile computing device, a tablet computing device, a desktop computing device, or a laptop computing device).

FIGS. 3A and 3B may represent an input screen. While input is effected using an input screen herein, it is understood that other methods of data entry (e.g., spoken instructions via a microphone) are also possible and are contemplated herein. FIG. 3A is an illustration of an input screen without any inputs yet entered, whereas FIG. 3B is an illustration of an input screen with inputs currently entered. The input screen may be used by a patient or a physician to input clinical-pathologic factors and/or a continuous multigene-expression score usable to calculate a risk score for the patient (e.g., a risk score associated with melanoma). Prior to reaching the input screen, a login screen may have been displayed using the risk score calculation application 300. The login screen may allow for the input of user login credentials (e.g., a username and password). Once entered, such user login credentials may be authenticated by the computing device 100 prior to advancing to the input screen. Validating the user login credentials may involve comparing the user login credentials to stored credentials associated with a plurality of authenticated users (e.g., to ensure that the present user is an authenticated user of the risk score calculation application 300 and/or to set permissions associated with the present user prior to proceeding with the risk score calculation, such as permissions related to which patient records are accessible by the present user). The stored credentials may be stored locally (e.g., within application data 112 in the data storage 108) and/or remotely (e.g., within a server that is accessible using an API).

As illustrated, the input screen may include a clinical-pathologic factors entry section 310 and a continuous multigene-expression profile score entry section 320. The clinical-pathologic factors entry section 310 may allow for the entry of various clinical-pathologic factors. For example, as illustrated in FIGS. 3A and 3B, patient age, patient gender, patient Breslow thickness (e.g., corresponding to a tumor Breslow thickness), patient ulceration level (e.g., corresponding to a tumor ulceration level), patient mitotic rate (e.g., corresponding to a tumor mitotic rate), and patient SLN status may all include fields in the clinical-pathologic factors entry section 310. Entry can occur in different methods. For example, an open text field/numeric field may be used (e.g., for patient's age). Alternatively, a drop-down calendar could be provided for patient's date of birth (based on which patient's age could be calculated). In some embodiments, as illustrated, drop-down menus with selectable options could be provided (e.g., options for level of ulceration corresponding to a tumor). Still further, radio buttons or sliders could also be used to enter clinical-pathologic data. Other data entry styles are possible and are contemplated herein. Additionally or alternatively, entry of other clinical-pathologic factors not pictured in FIGS. 3A and 3B are also contemplated herein. Even further, entry of the clinical-pathologic factors illustrated in FIGS. 3A and 3B using different data entry styles are also possible and contemplated herein. For example, a patient's age could be entered using a slider rather than a text/numeric entry field.

Although not illustrated in FIGS. 3A and 3B the units associated with any given input (if any), may be indicated on the input screen. Further, input into the clinical-pathologic factors entry section 310 may include an entry of units and/or a selection of units. For example, the mitotic rate may default to mitoses/mm². However, different input unit options may be selectable. Even further, as illustrated for Breslow thickness in FIGS. 3A and 3B, there may be options selectable for some or all clinical-pathologic factors to indicate that a certain clinical-pathologic factor is unknown and/or will not be provided. If this option is selected, that clinical-pathologic factor may be assigned an average value (e.g., average across a population or average value for other patients having similar values to the current patient for the remaining clinical-pathologic factors) and/or a risk score calculation may be performed without that clinical-pathologic value (e.g., the risk score calculation application 300 may generate a range of possible risk scores based on the entered clinical-pathologic factors).

Also as illustrated in FIGS. 3A and 3B, the input screen may include a continuous multigene-expression profile score entry section 320. As illustrated in FIG. 3A, the continuous multigene-expression profile score data field may not appear until, as illustrated in FIG. 3B, a selection is made that indicates that the patient has had a continuous multigene-expression profile score generated (e.g., and that one will be used in the risk score calculation). Once a selection is made to indicate that a continuous multigene-expression profile score will be provided (e.g., by selecting a check-box, as illustrated in FIG. 3B or a radio button), a data entry field may appear and be accessible. The continuous multigene-expression profile score may correspond to a continuous multigene-expression profile score used solely by the risk score calculation application 300 (e.g., a proprietary continuous multigene-expression profile score), in some embodiments.

Although not illustrated in FIGS. 3A and 3B, in some embodiments, the input screen of the risk score calculation application 300 may include a file upload section. The file upload section may allow a user (e.g., a physician) to upload a data file (e.g., from a server computing device or from the computing device 100) that includes clinical-pathologic factors and/or the continuous multigene-expression profile score. For example, a physician may upload a .pdf file, a .txt file, a .csv file, a .xml file, or another file format that includes a patient's clinical-pathologic factors (e.g., a laboratory report). This file may ultimately be read by the risk score calculation application 300 to extract the clinical-pathologic factors without the need for the user to manually enter the clinical-pathologic factors using the input screen. The risk score calculation application 300 reading the uploaded file may include performing optical character recognition (OCR), for example. In some embodiments, for example, the risk score calculation application 300 may receive an uploaded file that contains a continuous multigene-expression profile. Then, based on the continuous multigene-expression profile, the risk score calculation application 300 may calculate a continuous multigene-expression profile score for use in the risk score calculation.

Once the data entry in the clinical-pathologic factors entry section 310 and the continuous multigene-expression profile score entry section 320 is complete, a risk calculation button 330 may appear (e.g., as illustrated in FIG. 3B). The risk calculation button 330 may be engaged to cause the risk score calculation application 300 to determine a risk score or range of risk scores based on the data entered. In alternate embodiments, the risk calculation button 330 may always be present (e.g., regardless if all the data in the clinical-pathologic factors entry section 310 and the continuous multigene-expression profile score entry section 320 is entered), but if an input is received via the risk calculation button 330 that indicates to the risk score calculation application 300 to determine a risk score when some of the data has not been provided, the risk score calculation application 300 may, in various embodiments: (1) flag which entry fields still require data or (2) proceed to generate a range of scores based on ranges of possible values for those fields that remained empty prior to the risk calculation button 330 being engaged.

Regardless of how it is done, once the data has been obtained by the risk score calculation application 300, the risk score calculation application 300 may determine one or more risk scores based on the entered data. The one or more risk scores may correspond to various disease-related statistics. For example, in the case of melanoma, the risk score(s) may represent a SLN metastasis positivity, a RFS rate, a DMFS rate, a MSS rate, etc., or a combination thereof. The one or more risk scores may be calculated using a statistical model (e.g., a Cox regression model) and/or a machine-learned model (e.g., the machine-learned model 230 shown and described with reference to FIGS. 2A and 2B). Once the risk score(s) have been calculated, the may be output by the risk score calculation application 300. Outputting the risk score(s) may include inserting the risk scores into a clinical laboratory report and/or transmitting (e.g., as a pop notification or an email) the risk score(s) to a user of the computing device 100. Additionally or alternatively, outputting the risk score(s) may include displaying the risk score(s) on an output screen, as illustrated in FIG. 4.

The output screen illustrated in FIG. 4 includes a risk score range output section 410 and a single risk score output section 420. The risk score range output section 410 may include a range of risk scores (e.g., based only on the clinical-pathologic factors and not accounting for the continuous multigene-expression profile score). Such a range may be output when the continuous multigene-expression profile score is not provided to the risk score calculation application 300 using the input screen of FIGS. 3A and 3B, for example. Alternatively, as illustrated in FIG. 4, the range of risk scores may be provided in the risk score range output section 410 as a frame of reference for the risk score provided in the single risk score output section 420. While the range of risk scores provided in the risk score range output section 410 in FIG. 4 corresponds to a range of risk scores based only on clinical-pathologic factors (and not the continuous multigene-expression profile score), it is understood that other ways of calculating the range of risk scores are possible and are contemplated herein. For example, a subset of the clinical-pathologic factors may be combined with the continuous multigene-expression profile to generate a range of risk scores for unknown clinical-pathologic factors (e.g., for unknown SLN status, as illustrated in FIG. 3B) or to demonstrate what a change in certain clinical-pathologic factors (e.g., patient weight or smoking history) could mean for risk score. Further, in some embodiments where some of the clinical-pathologic factors are unknown and/or not provided and/or where the continuous multigene-expression profile is unknown or not provided, only the range of risk scores may be displayed (i.e., the single risk score output section 420 may not be displayed).

While the range of risk scores provided in the risk score range output section 410 and the risk score provided in the single risk score output section 420 are presented in FIG. 4 as text, it is understood that other presentations are also possible and are contemplated herein. For example, the range of risk scores could be displayed as a graph or chart (e.g., a bar chart, a pie chart, a histogram, etc.). Likewise, the single risk score could be depicted as a single point on the chart (e.g., within the range outlined by the range of risk scores). Other depictions of the range of risk scores and/or the individual risk score are possible and are contemplated herein.

In some embodiments, along with displaying the risk score or range or risk scores, the risk score calculation application 300 may also provide context along with the risk score(s). For example, the risk score calculation application 300 may provide an indication of additional diagnostic or treatment steps recommended to be taken based on the score (e.g., a recommendation that a SLNB be performed based on the score).

In addition, in some embodiments, once the risk score(s) have been determined, in addition to or instead of displaying the results, the risk score(s) may be stored. For example, the risk score(s) may be saved locally (e.g., as application data 112 within the data storage 108 of the computing device 100) and/or remotely (e.g., within a cloud server) for later access. Oppositely, in some embodiments, the risk score(s) may explicitly not be stored (e.g., to avoid the risk score calculation application 300 retaining personal health information (PHI)).

After outputting the risk score(s), the risk score calculation application 300 may obtain additional data (e.g., via a user interface 104 of the computing device 100). The additional data may include additional or revised clinical-pathological factors for the patient and/or a continuous multigene-expression profile score (if one wasn't provided in the first place) or a revised continuous multigene-expression profile score. This additional data may have be gathered (e.g., by a physician, pathologist, patient, etc.) based on an indication (e.g., output to a display of the user interface 104 of the computing device 100) by the risk score calculation application 300 that additional diagnostics be performed based on the risk score(s) previously calculation. For example, the risk score calculation application 300 may have displayed an indication based on a calculated risk score (or range of calculated risk scores) that a SLNB was to be performed. Thereafter, the physician may have recommended to the patient that the patient receive an SLNB, the results of the SLNB may have been measured by a pathologist, and the pathologist may enter the results as additional clinical-pathologic factors into the risk score calculation application 300. Obtaining additional data after the original risk score calculation may happen at a supplementary input screen of the risk score calculation application 300, for example. The supplementary input screen may look similar to the input screen illustrated in FIG. 3B, for example, with all the previous data obtained by the risk score calculation application 300 being prepopulated into the respective fields.

Upon obtaining additional or revised data (e.g., additional or revised clinical-pathologic factors or an additional or revised continuous multigene-expression profile score), the risk score calculation application 300 may determine one or more revised risk scores. The revised risk score(s) may be determined using the same statistical model and/or machine-learned model as the original risk score(s) and/or a different statistical model and/or machine-learned model, in various embodiments.

### III. Example Processes

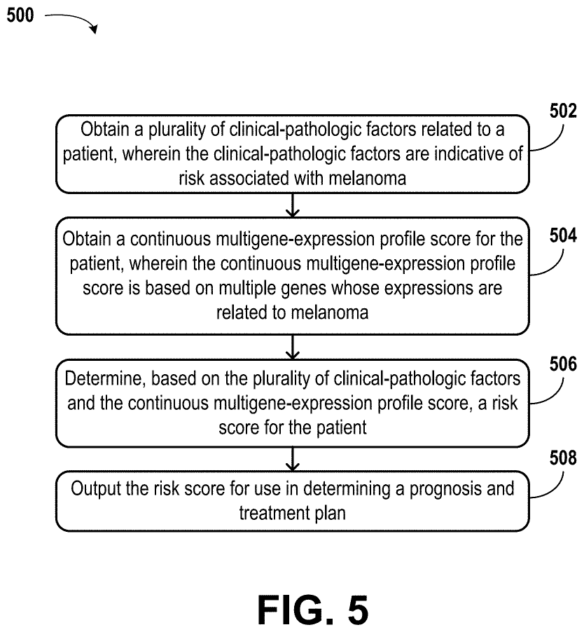

FIG. 5 is a flowchart diagram of a method 500, according to example embodiments. In some embodiments, the method 500 may be performed by a computing device (e.g., the computing device 100 shown and described with reference to FIG. 1). For example, the computing device 100 may include a non-transitory, computer-readable medium (e.g., data storage 108) with instructions (e.g., instructions 118) stored thereon. The instructions may be executable by a processor (e.g., processor 106) to execute the method 500.

At block 502, the method 500 may include obtaining a plurality of clinical-pathologic factors related to a patient. The clinical-pathologic factors may be indicative of risk associated with melanoma (or some other cancer or disease).

At block 504, the method 500 may include obtaining a continuous multigene-expression profile score for the patient. The continuous multigene-expression profile score may be based on multiple genes whose expressions are related to melanoma (or some other cancer or disease).

At block 506, the method 500 may include determining, based on the plurality of clinical-pathologic factors and the continuous multigene-expression profile score, a risk score for the patient.

At block 508, the method 500 may include outputting the risk score for use in determining a prognosis and treatment plan.

In some embodiments of the method 500, block 504 may include receiving a continuous multigene-expression profile for the patient based on multiple genes whose expressions are related to melanoma. Block 504 may also include calculating the continuous multigene-expression profile score based on the continuous multigene-expression profile.

In some embodiments of the method 500, the continuous multigene-expression profile score may include a score between 0 and 1 that represents expressions of 31 different genes relating to melanoma (or some other cancer or disease).

In some embodiments, the method 500 may also include obtaining, after block 508, one or more additional clinical-pathologic factors related to the patient. Additionally, the method 500 may include calculating, based on the plurality of clinical-pathologic factors, the one or more additional clinical-pathologic factors, and the continuous multigene-expression profile score, a revised risk score for the patient. Further, the method 500 may include outputting the revised risk score for use in determining a prognosis and treatment plan.

In some embodiments of the method 500, block 508 may include generating a clinical laboratory report usable for patient care. Further, block 508 may include causing an associated printing device to print the clinical laboratory report.

In some embodiments of the method 500, the plurality of clinical-pathologic factors may include an age of the patient, a gender of the patient, a tumor site location, a histologic type, a Breslow thickness measurement, a transected base measurement, an ulceration measurement, a microsatellites measurement, a mitotic rate, a lymphovascular invasion measurement, a tumor infiltrating lymphocytes measurement, a tumor regression, a sentinel lymph node status, and/or an in-transit disease/satellites measurement.

In some embodiments of the method 500, the risk score may include a SLN metastasis positivity, a RFS rate, a DMFS rate, or a MSS rate.

In some embodiments, the method 500 may also include receiving user login credentials. Further, the method 500 may include validating the user login credentials by comparing the user login credentials to stored credentials associated with a plurality of authenticated users.

In addition, the plurality of authenticated users may include physicians or clinicians permitted to provide and access information associated with the patient.

Additionally or alternatively, the plurality of authenticated users may include the patient.

In some embodiments of the method 500, block 508 may include providing the risk score to an electronic health record associated with the patient.

In some embodiments of the method 500, the plurality of clinical-pathologic factors may be received from user input into a browser-based application. In addition, the continuous multigene-expression profile score for the patient may be received from user-input into the browser-based application. Further, block 508 may include displaying the risk score via the browser-based application.

In some embodiments of the method 500, the plurality of clinical-pathologic factors may be received from user input into a mobile application. In addition, the continuous multigene-expression profile score for the patient may be received from user input into the mobile application. Further, block 508 may include causing an associated user interface to display the risk score via the mobile application.

In some embodiments, the method 500 may also include determining, based on the plurality of clinical-pathologic factors, a range of risk scores for use in determining a prognosis and treatment plan. Further, the method 500 may include outputting the range of risk scores.

In some embodiments of the method 500, block 506 may include applying a machine-learned model to the plurality of clinical-pathologic factors and the continuous multigene-expression profile score.

Further, the machine-learned model may include an artificial neural network. In addition, applying the machine-learned model to the plurality of clinical-pathologic factors may include applying machine-learned weights of the artificial neural network to each of the clinical-pathologic factors and the continuous multigene-expression profile score.

In some embodiments of the method 500, block 506 may include applying a statistical model (e.g., a Cox regression model) to the plurality of clinical-pathologic factors and the continuous multigene-expression profile score.

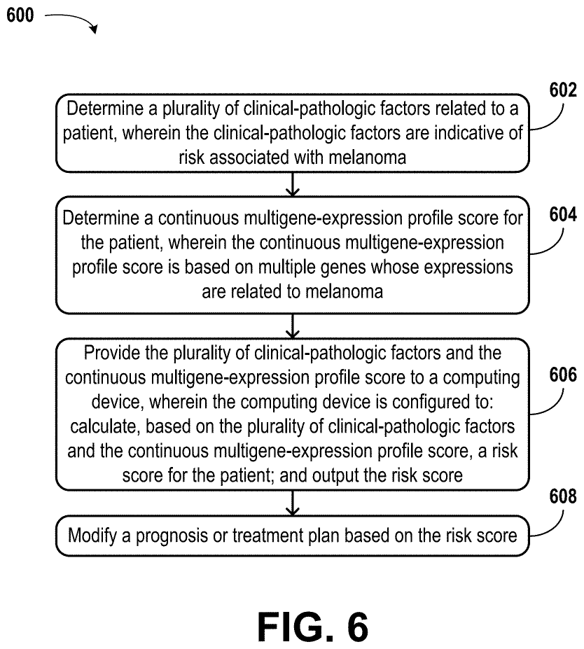

FIG. 6 is a flowchart diagram of a method 600, according to example embodiments. In some embodiments, the method 600 may be performed, in part, using a computing device (e.g., the computing device 100 shown and described with reference to FIG. 1). For example, a physician, clinician, pathologist, oncologist, etc. may use the computing device 100 to perform the method 600.

At block 602, the method 600 may include determining a plurality of clinical-pathologic factors related to a patient. The clinical-pathologic factors may be indicative of risk associated with melanoma (or some other cancer or disease).

At block 604, the method 600 may include determining a continuous multigene-expression profile score for the patient. The continuous multigene-expression profile score may be based on multiple genes whose expressions are related to melanoma (or some other cancer or disease).

At block 606, the method 600 may include providing the plurality of clinical-pathologic factors and the continuous multigene-expression profile score to a computing device. The computing device may be configured to calculate, based on the plurality of clinical-pathologic factors and the continuous multigene-expression profile score, a risk score for the patient. The computing device may also be configured to output the risk score.

At block 608, the method 600 may include modifying a prognosis or treatment plan based on the risk score.

In some embodiments of the method 600, block 608 may include determining that further diagnostic testing is to be performed or performing further diagnostic testing.

In some embodiments of the method 600, block 608 may include performing a SLN biopsy on the patient. In addition, the method 600 may include providing results from the SLN biopsy on the patient to the computing device. The computing device may be further configured to calculate, based on the plurality of clinical-pathologic factors, the results from the SLN biopsy, and the continuous multigene-expression profile score, a revised risk score for the patient. Additionally, the computing device may be configured to output the revised risk score.

In some embodiments of the method 600, block 604 may include providing a continuous multigene-expression profile to the computing device. Additionally, the computing device may be further configured to calculate the continuous multigene-expression profile score based on the continuous multigene-expression profile.

In some embodiments of the method 600, block 602 may include performing one or more laboratory tests using one or more samples from the patient, receiving demographic information from the patient, or accessing one or more records associated with the patient.

### Example 1. Using 31-Gene Expression Profiling to Personalize Risk of Recurrence and Metastasis Prognosis in Patients with Cutaneous Melanoma

Background: The National Comprehensive Cancer Network recommends patient management strategies based on the American Joint Committee on Cancer (AJCC) staging system derived from binned histopathologic data and fails to report personalized outcomes. The 31-gene expression profile (31-GEP) test examines tumor biology for precise risk prediction and complements clinicopathologic features. Objective: To develop and validate an integrated algorithm (i31-GEP) that combines the continuous 31-GEP score with clinicopathologic features for use as a personalized outcomes prediction tool. Methods: A multivariable Cox regression model using patient clinicopathologic features and continuous 31-GEP scores (N=918) was used to develop precise risk predictions for RFS and DMFS. The algorithm was validated in a cohort of 305, and the net reclassification analysis was performed. Results: The 31-GEP score was the strongest predictor of RFS (HR 5.5% CI 1.33-25.59], P<0.001) and DMFS (HR 6.74 [95% CI 1.13-39.94], P<0.001). The i31-GEP returned risk predictions in line with the range of AJCC observed outcomes and improved classification of risk of melanoma recurrence over AJCC staging (P=0.003). Conclusions: The i31-GEP improves precision of recurrence-free and metastasis-free survival prediction over AJCC staging that may lead to personalized, risk-aligned management strategies.

### Methods

### Algorithm Development

A cohort of 1223 CM patients from a previously published meta-analysis combining two retrospective and one prospective cohort was used to develop (N=918, 75%) and validate (N=305, 25%) a Cox regression model integrating the continuous 31-GEP score with relevant clinicopathologic features (i31-GEP) to develop a risk prediction algorithm for RFS (recurrence-free survival; where a recurrence is considered a regional event occurring 4 months or more after diagnosis or a distant metastasis) and DMFS (distant metastasis-free survival). Covariates include continuous variables of the 31-GEP score, Breslow thickness, mitotic rate, and age, and the binary variables of ulceration and SLN status.

### Statistical Analyses and Model Validation

Comparison between cohort characteristics was performed using the Pearson's Chi-squared test or Wilcoxon Rank Sum test where appropriate. Recurrence predictions and outcomes were compared between the i31-GEP and AJCC stage using Pearson's Chi-squared test with Yates' continuity correction. Decision curve analysis was performed to assess the net benefits of the i31-GEP compared to AJCC staging. To increase model accuracy, Breslow thickness and the 31-GEP score underwent log and p-spline transformations, respectively. In all cases, P<0.05 was deemed to be statistically significant.

### Results

### Patient Demographics

Patient characteristics for the training and validation cohorts can be found in Table 1. The median age for the training and validation cohorts was 58 years (range: 18-94 years) and 59 years (range: 18-92 years), respectively (P=0.492). No significant differences were found for the training vs. validation cohort for the median mitotic rate (1/mm2 [range 0-78] vs. 1/mm2 [range 0-74], P=0.798), presence of ulceration (26.1% vs. 26.2%, P=0.976), Breslow thickness (1.3 mm [range 0.1-29.0 mm] vs. 1.3 mm [range 0.2-13.0 mm], P=0.360), SLN positivity (24.7% vs. 27.9%, P=0.276), median 31-GEP score (0.42 [range 0-1] vs. 0.40 [range 0-1], P=0.902), recurrence (24.8% vs. 24.3%, P=0.840), or distant metastasis (18.2% vs. 16.4%, P=0.476). Also, there was no significant difference in the number of patients in each AJCC stage (P=0.252) or T-category (P=0.382).

### Model Performance

The 31-GEP score was the strongest predictor of RFS (HR 5.84 [95% CI 1.33-25.59], P<0.001) and DMFS (HR 6.74 [95% CI 1.13-39.94], P<0.001) within the model, and was independent of clinicopathologic features (Table 2). Older age, increased Breslow thickness, ulceration, increasing mitotic rate, and a positive SLN were also significant predictors of a lower 3-year RFS and DMFS within the model (Table 2).

As an indicator of model prediction accuracy, the i31-GEP model predicted 3-year RFS and DMFS rates comparable to the actual risk observed by KM analysis in the cohort, with the average estimated risk for each AJCC substage being within the confidence intervals obtained from the KM analysis. The i31-GEP prediction was significantly more accurate than AJCC v8 staging for RFS (P=0.030) (Table 3). Risk estimates for RFS produced a relative reduction in prediction error of 32.3% compared with the AJCC stage risk estimates for RFS.

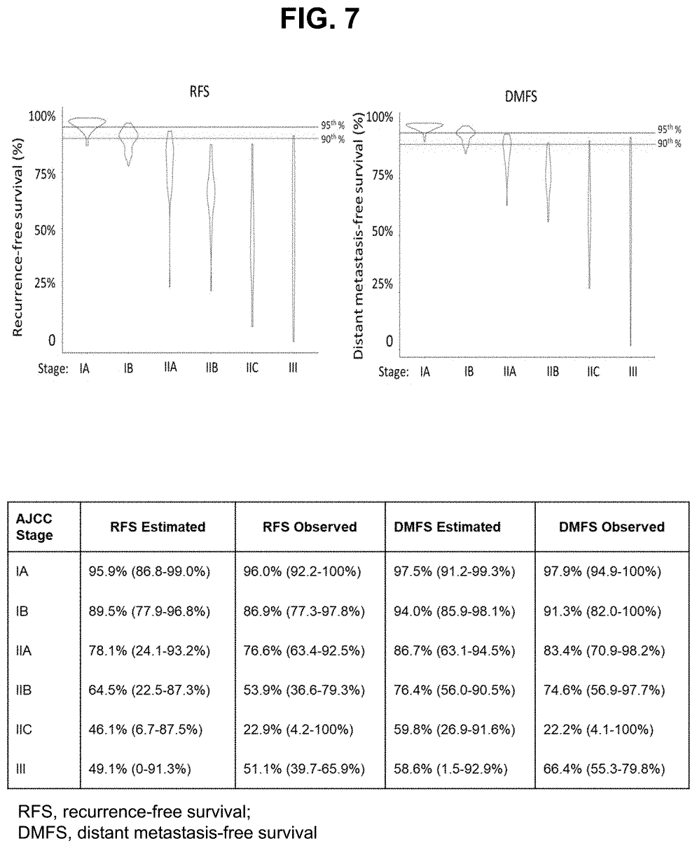

Current staging criteria uses Breslow thickness, ulceration, and SLN status alone to bin patients into generalized MSS risk prediction categories that do not fully capture the variability of survival outcomes seen in the clinic. To improve survival risk prediction accuracy and personalization, the i31-GEP model was developed combining the continuous 31-GEP score in conjunction with clinicopathologic features. The 31-GEP was the strongest predictor for RFS and DMFS (Table 2), and the model accurately predicted survival outcomes well within the confidence intervals of observed data produced in KM analysis (FIG. 7, Table 3). Finally, the model reduced the number of potential interventions compared with AJCC staging. These results suggest that the 31-GEP score adds significant prognostic value to clinicopathologic feature assessment for a personalized risk prediction that may lead to more individualized, risk-aligned patient management strategies.

The i31-GEP refines risk prediction for melanoma recurrence and removes intra-stage variation in the current AJCC staging system, to provide a more precise, individualized risk estimate that may help personalize patient management.

TABLE 1
Demographics

| | | | | |
|---|---|---|---|---|
| Descriptor | Training data | Validation data | Combined | |
| (N) | (n = 918) | (n = 305) | (n = 1223) | P-value |

Age (1223)

| | | | | | | | |
|---|---|---|---|---|---|---|---|
| Median (Range) | 59 | (18-94) | 59 | (18-92) | 59 | (18-94) | .492 |

| Mitotic Rate (1223) | | | | | | | |
|---|---|---|---|---|---|---|---|
| Median (Range) | 1 | (0-78) | 1 | (0-74) | 1 | (0-78) | .798 |

| Ulceration (1223) | | | | | | | |
|---|---|---|---|---|---|---|---|
| no | 678/918 | (73.86%) | 225/305 | (73.77%) | 903/1223 | (73.83%) | .976 |
| yes | 240/918 | (26.14%) | 80/305 | (26.23%) | 320/1223 | (26.17%) | |
| Breslow (1218) | | | | | | | |
| Median (Range) | 1.3 | (0.1-29.0) | 1.3 | (0.2-13.0) | 1.3 | (0.1-29.0) | .360 |
| Node Positive (1223) | | | | | | | |
| no | 691/918 | (75.27%) | 220/305 | (72.13%) | 911/1223 | (74.49%) | .276 |
| yes | 227/918 | (24.73%) | 85/305 | (27.87%) | 312/ 1223 | (25.51%) | |

| GEP Continuous (1223) | | | | | | | |
|---|---|---|---|---|---|---|---|
| Median (Range) | 0.42 | (0-1) | 0.40 | (0-1) | 0.41 | (0-1) | .902 |
| AJCC Stage, 8^{th} ed. (1223) | | | | | | | |
| Stage IA | 298/918 | (32.46%) | 107/305 | (35.08%) | 405/1223 | (33.12%) | .252 |
| Stage IB | 183/918 | (19.93%) | 43/305 | (14.1%) | 226/1223 | (18.48%) | |
| Stage IIA | 103/918 | (11.22%) | 37/305 | (12.13%) | 140/1223 | (11.45%) | |
| Stage IIB | 72/918 | (7.84%) | 25/305 | (8.2%) | 97/1223 | (7.93%) | |
| Stage IIC | 34/918 | (3.7%) | 7/305 | (2.3%) | 41/1223 | (3.35%) | |
| Stage III | 227/918 | (24.73%) | 85/305 | (27.87%) | 312/1223 | (25.51%) | |
| NULL | 1/918 | (0.11%) | 1/305 | (0.33%) | 2/1223 | (0.16%) | |
| T-Category (1218) | | | | | | | |
| T1a | 230/914 | (25.16%) | 83/304 | (27.3%) | 313/1218 | (25.7%) | .382 |
| T1b | 123/914 | (13.46%) | 42/304 | (13.82%) | 165/1218 | (13.55%) | |
| T2a | 197/914 | (21.55%) | 53/304 | (17.43%) | 250/1218 | (20.53%) | |
| T2b | 61/914 | (6.67%) | 26/304 | (8.55%) | 87/1218 | (7.14%) | |
| T3a | 100/914 | (10.94%) | 37/304 | (12.17%) | 137/1218 | (11.25%) | |
| T3b | 82/914 | (8.97%) | 32/304 | (10.53%) | 114/1218 | (9.36%) | |
| T4a | 47/914 | (5.14%) | 16/304 | (5.26%) | 63/1218 | (5.17%) | |
| T4b | 74/914 | (8.1%) | 15/304 | (4.93%) | 89/1218 | (7.31%) | |
| Recurrence (1223) | | | | | | | |
| No | 690/918 | (75.16%) | 231/305 | (75.74%) | 921/1223 | (75.31%) | .840 |
| Yes | 228/918 | (24.84%) | 74/305 | (24.26%) | 302/1223 | (24.69%) | |
| Distant Metastasis (1223) | | | | | | | |
| No | 751/918 | (81.81%) | 255/305 | (83.61%) | 1006/1223 | (82.26%) | .476 |
| Yes | 167/918 | (18.19%) | 50/305 | (16.39%) | 217/1223 | (17.74%) | |

TABLE 2
Multivariable Cox regression model integrating 31-GEP
and clinicopathologic features
for 3-year risk of cutaneous melanoma recurrence.

| 3-year RFS | | | | 3-year DMFS | | | |
|---|---|---|---|---|---|---|---|
| | HR | | P- | | HR | | P- |
| Feature | (95% CI) | | value | Feature | (95% CI) | | value |
| Age [symbol] | 1.01 | (1.00-1.02) | .006 | Age [symbol] | 1.01 | (1.00-1.02) | .02 |
| Breslow [symbol] | 1.91 | (1.58-2.31) | <.001 | Breslow [symbol] | 1.79 | (1.43-2.45) | <.001 |
| Ulceration | 1.37 | (1.03-1.84) | .033 | Ulceration | 1.76 | (1.24-2.47) | .001 |
| Mitotic Rate [symbol] | 1.03 | (1.01-1.04) | <.001 | Mitotic Rate [symbol] | 1.02 | (1.00-1.04) | .008 |
| SLN Status | 2.83 | (2.14-3.75) | <.001 | SLN Status | 3.24 | (2.33-4.50) | <.001 |
| 31-GEP [symbol] | 5.84 | (1.33-25.59) | <.001 | 31-GEP [symbol] | 6.74 | (1.13-39.94) | <.001 |

[symbol]

Indicates continuous variables.
^{#}To improve the model’s accuracy, Breslow thickness underwent log transformation and the 31-GEP continuous score underwent p-spline transformation.
The 31-GEP HR value represents the maximum P-spline value for the 31-GEP. 31-GEP, 31-gene expression profile; RFS, recurrence-free survival; DMFS, distant metastasis-free survival; HR, hazard ratio

TABLE 3
Reclassification of risk by the i31-GEP
compared to current AJCC staging

| | Increased risk | | Decreased risk | | | |
|---|---|---|---|---|---|---|
| | predicted | | predicted by | | Net | |
| | by model, | | model, | | reclassification | |
| | N (%)^{#} | | N (%)^{#} | | improvement | |
| | 3-year RFS | | | | | |
| Events | 39 | (34%) | 34 | (18%) | 5 | (16%) |
| Non-events | 77 | (66%) | 154 | (82% | 77 | (16%) |
| Total | 116 | (100%) | 188 | (100%) | 82 | (32%) |
| | 3-year DMFS | | | | | |
| Events | 27 | (20%) | 22 | (13%) | 5 | (7%) |
| Non-events | 105 | (80%) | 150 | (87%) | 45 | (7%) |
| Total | 132 | (100%) | 172 | (100%) | 82 | (14%) |

^{#}Relative to AJCC v8 Stage
RFS, recurrence-free survival; DMFS, distant metastasis-free survival

### Example 2. Integration of the Continuous 31-Gene Expression Profile Score and Clinicopathologic Features to Predict Sentinel Lymph Node Status in Patients with Cutaneous Melanoma

Background: National guidelines recommend that sentinel lymph node biopsy (SLNB) be offered to patients with a positivity risk >10% (T2-T4 tumors). Patients with T1a tumors and no high-risk features have a <5% risk of SLN positivity and can forego SLNB. However, the decision to perform SLNB is less certain for patients with a positivity risk of 5-10% (T1a tumors with high-risk features or a T1b tumor). This disclosure demonstrates that integrating clinicopathologic features with results of the prognostic 31-gene expression profile (31-GEP) test using advanced artificial intelligence techniques provides a more individualized SLN risk prediction. Methods: An integrated 31-GEP (i31-GEP) neural network algorithm incorporating clinicopathologic features and the continuous 31-GEP score was developed on a previously reported cohort (N=1398) and validated on an independent cohort (N=1674). Results: Compared to clinicopathologic features, the continuous 31-GEP score had the largest likelihood ratio (G2=91.3, P<0.001) and the highest importance in predicting SLN positivity. The i31-GEP increased the percentage of patients with T1-T4 tumors predicted to have low (<5%) SLN-positive risk from 8.5% to 27.7%. Importantly, for patients originally classified with 5-10% SLN positivity risk (eligible T1a and T1b), i31-GEP re-classified 63% of patients whose true risk was <5% or >10%. Conclusions: The i31-GEP model demonstrated a high concordance between predicted and observed SLN positivity rates. The i31-GEP could be used to identify patients with a risk under the 5% threshold for performance of SLNB set by national guidelines and focus healthcare resources on patients more likely to have a positive SLN (>10%) while reducing uncertainty (SLN positive risk from 5-10%) in the eligible T1 population

Up to 88% of sentinel lymph node biopsies on patients with cutaneous melanoma are negative, providing little benefit while exposing the patient to surgical risks. Consequently, an unmet clinical need is an improved method for predicting the risk of sentinel node (SLN) positivity, particularly in patients with thin (T1a with high-risk features or T1b) tumors with less certain SLN positivity risk (5-10%). An advanced artificial intelligence algorithm was developed and validated that integrates molecular gene expression from the 31-gene expression profile (31-GEP) with relevant clinicopathologic factors to predict SLN positivity risk in patients with T1 -T4 cutaneous melanoma (i31-GEP). The i31-GEP result re-classified 63% of cases with SLN positivity risk between 5 and 10% to <5% or >10% risk. More accurate sentinel node status prediction can provide necessary guidance to direct healthcare resources to patients at high-risk for sentinel node positivity. The data provided in this study give an opportunity for more precise, risk-aligned patient care.

### Methods:

### Patient Demographics

Development Cohort

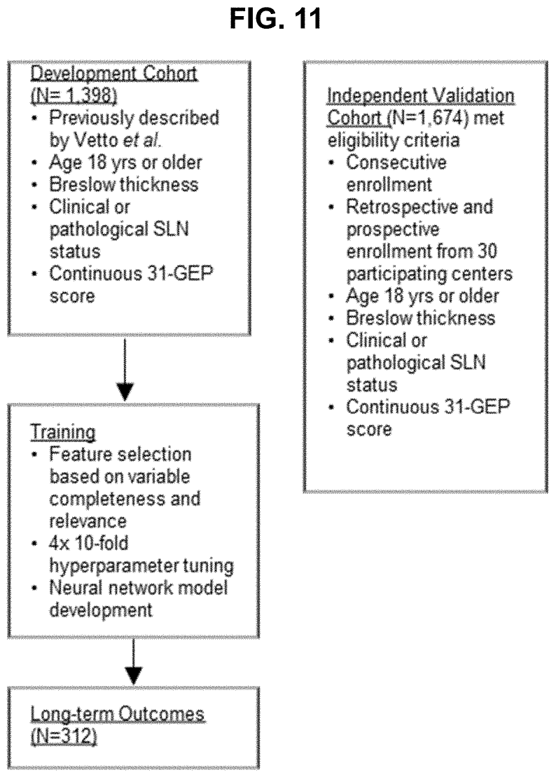

The training cohort has been previously described. The model was trained on 1398 patients who were ≥18 years of age with primary tumors of known Breslow thickness (T1 -T4), a continuous 31-GEP test result, and either clinically (287/1398; 20.5%) or pathologically (1111/1398; 79.5%) known SLN status (FIG. 11).

Validation Cohort

A total of 1674 consecutively tested patients with a continuous 31-GEP test result were enrolled under one of four IRB-approved studies from 25 surgical and five dermatological centers. Eligibility criteria were the same as for the training cohort (FIG. 11).

### 31-GEP Testing

The 31-GEP test (DecisionDx-Melanoma, Castle Biosciences, Inc., USA) was used to analyze the expression of 28 prognostic genes and three control genes from primary CM tumors, as previously described. All 31-GEP testing was performed in a CAP-accredited and CLIA-certified laboratory.

i31-GEP Development and Validation

Data collected for analysis and i31-GEP algorithm training included the continuous variables of the 31-GEP score, Breslow thickness, MR, and age, and the categorical variables of ulceration status, tumor regression, LVI, tumor-infiltrating lymphocytes (TILs), age, sex, microsatellites, histopathologic subtype, transected bases, and tumor site. Regression, MR, microsatellites, and ulceration were imputed to “absent” if not indicated in the patient records, consistent with CAP synoptic reporting guidelines. Models were generated in the R v3.6.3 using the caret package to generate neural networks with the nnet submodule and four times ten-fold cross-validation for hyperparameter selection. Because neural network algorithms are subject to overfitting with the inclusion of excess variables that do not contribute to the algorithm, variable selection is an important aspect of neural network development; therefore, variables occurring in <5% (microsatellites, and LVI) of cases or those with insufficient completeness due to non-standardized variable reporting (TILs) of the training cohort were excluded. Next, multiple iterations of the model were run with the remaining features to determine which contributed significantly to the prediction algorithm. Nodal events were coded as 0 for negative or 1 for positive to generate a regression algorithm.

Validation of the algorithm was performed on an independent cohort of eligible patients with T1-T4 tumors (N=1674) as described above. Patients with T1a disease with documentation of MR ≥2/mm2, presence of LVI, absence of TILs, age <40 years, presence of microseatellites, presence of regression, or transected base were categorized as having high-risk T1a tumors (T1a-HR). Patients with T1a tumors and none of those features specified were considered low-risk T1a (T1a-LR), while patients with T4 tumors have >25% SLN positivity risk, and are unlikely to forego SLNB, they were included in the algorithm training and validation to determine if risk stratification even in high-risk tumors can be achieved.

Accuracy metrics were calculated by assigning i31-GEP predictions of <5% as a negative and ≥5% SLN positivity risk as a positive result. SLNB reduction rate was calculated by dividing the number of negative test results by the full population, and % yield was calculated as the proportion of true positive test results among all test results (PPV).

### Statistical Analysis

The importance of each variable contributing to the i31-GEP algorithm was assessed using the default variable importance assessment functions included in the caret package for neural network models (R package v3.6.3). An SLN positivity risk of <5% was considered low risk, between 5-10% indeterminant risk, and >10% was considered high-risk in concordance with NCCN guidelines for the performance of SLNB. Comparison of clinicopathologic feature prevalence between cohorts was performed using the Mann-Whitney U test or Fisher's exact test. A P value <0.05 was considered statistically significant. Continuous variables are reported as median (range), and dichotomous variables as a percentage (n/N). Kaplan-Meier analysis and the log-rank test were used to compare survival outcomes. Simple logistic regression was performed to show the probability of a positive SLN for each variable within the training cohort; continuous variables are plotted as a logistic regression line with 95% confidence intervals (95% CI), and binary variables are plotted as mean SLN positivity with 95% CI.

### Results:

### Patient Demographics

The i31-GEP algorithm was trained on a cohort previously described by Vetto et al. (“Guidance of sentinel lymph node biopsy decisions in patients with T1-T2 melanoma using gene expression profiling.” Future Oncol Lond Engl. 2019 April; 15(11):1207-17); the validation cohort is a previously unreported novel cohort (N=1674) (FIG. 11, Table 4). Demographics revealed a significant difference between the development and validation cohorts in median GEP score (0.35 [range 0-1] vs. 0.40 [range 0-1], P<0.001), age (63 years [range 18-101] vs. 65 years [range 21-97]; P<0.001) and MR (1.0/mm2 [range 0-63.0/mm2] vs. 1.0/mm2 [range 0-74.0/mm2]; P<0.001), and the number of patients with an absence of TILs (13.3% vs. 9.9%, P=0.003), presence of microsatellites (0.4% vs. 1.1%, P=0.022), and transected base (19.5% vs 34.9%; P<0.001). There was no significant difference between cohorts for sex (P=0.537), Breslow thickness (P=0.292), ulceration (P=0.195), or SLN positivity (P=0.368) (Table 4).

i31-GEP Algorithm Development and Specification

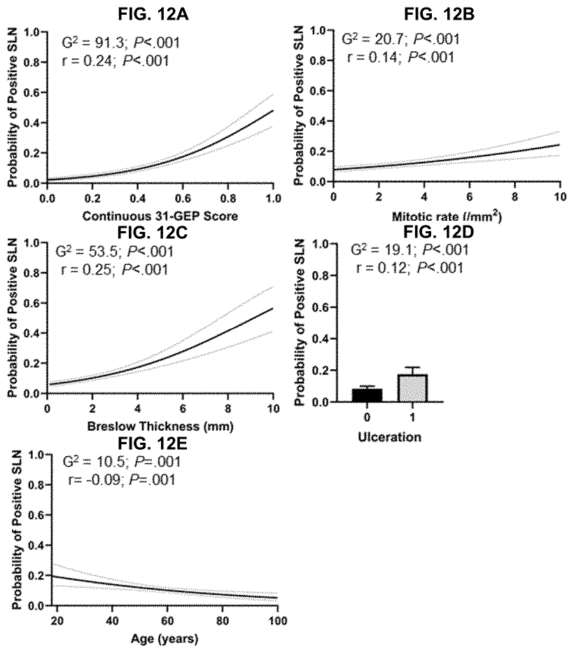

Features that significantly contributed to the model, as described in the methods, were included in i31-GEP development and included the continuous variables of 31-GEP, Breslow thickness, MR, and age, and the binary variable of ulceration. Variable importance assessment functions determined that the 31-GEP score had the highest importance (100 on a scale of 0-100), followed by MR (46), Breslow thickness (37), ulceration (21), and age (21) (Table 7). Logistic regression of variables within the training cohort is shown in FIG. 12 and Table 7. The 31-GEP had the highest log-likelihood value (G2=91.3; P<0.001), indicating that it is the best predictor of SLN positivity followed by Breslow thickness (G2=53.5; P<0.001).

i31-GEP Performance

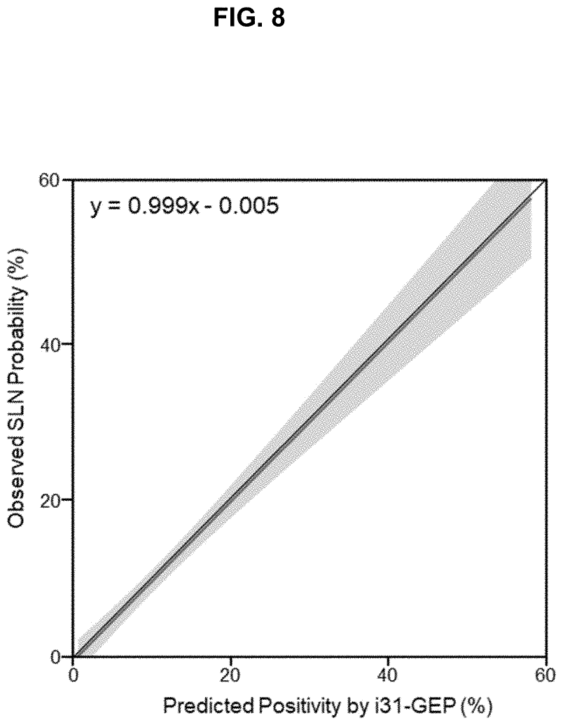

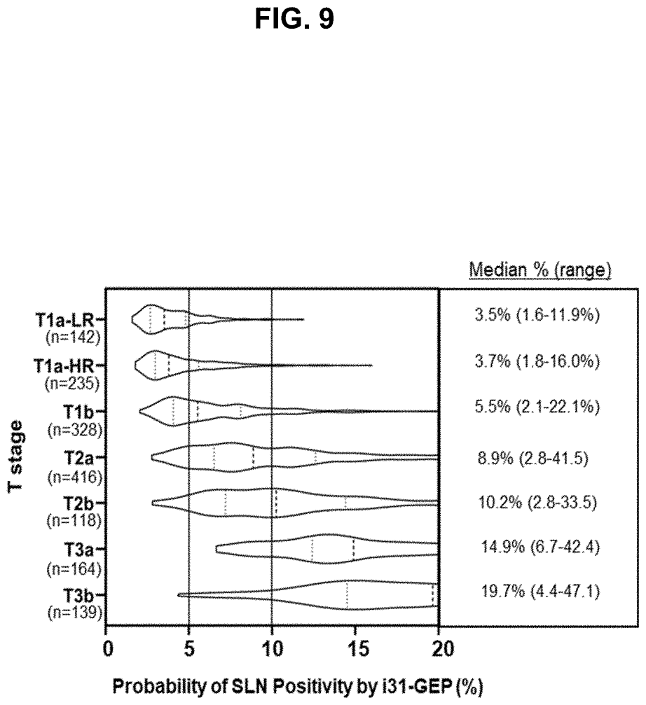

Validation in an independent cohort of N=1674 patients with T1-T4 tumors demonstrated alignment between observed SLN positivity rates compared to i31-GEP predictions with a slope of 1.0 demonstrated by linear regression (FIG. 8). Moreover, the i31-GEP model predicted that 27.7% (464/1674) of patients had a predicted probability of <5%, and 41.6% (696/1674) had a predicted probability of >10% compared with just 8.5% with <5% SLN positivity risk for a low-risk T1a designation. In the validation cohort, 377 tumors were designated as T1a (235 of which had one or more high-risk features), and 328 as T1b. The i31-GEP re-classified 68.5% (161/235) of T1a tumors with at least one high-risk feature, and 40.9% (134/328) of T1b as low risk (<5% risk of SLN positivity) for a total of 52.4% of higher-risk T1 tumors re-classified as <5% risk. Moreover, it re-classified 4.7% (11/235) of patients with a T1a tumor and at least one high-risk feature and 14.3% (47/328) T1b tumors as having >10%, risk re-classifying a total of 10.3% of higher-risk T1 tumors as having a predicted risk >10%. In sum, of the 563/1542 patients with SLN positivity risk classified by T-stage as between 5-10%, the i31-GEP re-classified 62.7% (353/563) to <5% or >10% SLN positivity risk (FIG. 9, Table 5). Similarly, validation of cases in the T2 population demonstrated that 12.5% (52/416) of T2a tumors and 4.2% (5/118) of patients with T2b tumors were predicted to have a <5% risk and 44.7% (186/416) of T2a and 44.1% (52/118) of T2b cases had a 5-10% risk of SLN positivity, providing potentially meaningful risk reduction within T2 tumors while identifying more precise risk for those T2 cases with a >10% risk of SLN positivity (FIG. 9, Table 5).

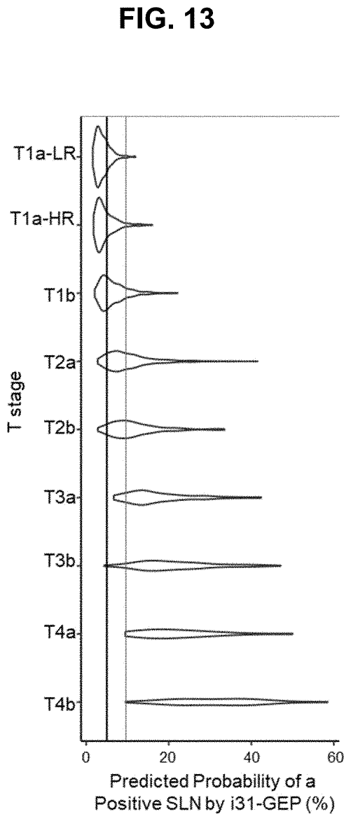

On the other hand, while only 0.3% (1/303) of T3 cases had a <5% risk prediction, 10.2% (31/303) of cases had a risk between 5-10% with the majority of T3 cases having a risk >10% as expected. Validation in patients with T4 tumors confirmed that while the majority (96%) had SLN positivity predictions higher than 10%, the range was wide (9.5-58%; Table 5, FIG. 13), which may be important in SLNB discussions for patients with comorbidities in which the benefit/risk ratio of SLNB is concerning. Overall validation demonstrated that the i31-GEP improved precision of risk predictions over T stage alone.

i31-GEP Accuracy

To assess the accuracy of the i31-GEP, a predicted risk <5% was considered a negative test, and a ≥5% risk was considered a positive test per national guidelines. The T1a low-risk population had no positive SLNs, while the T3 population only had one negative test result, and the T4 population had no negative results; therefore, accuracy was restricted to the eligible T1 and T2 populations. The i31-GEP had an overall high negative predictive value (97.4%) and a high sensitivity (89.8%), indicating a low false-negative rate. Based on the low risk of SLN positivity with a negative i31-GEP result, the procedure reduction rate (32.1% overall) was calculated as the proportion of negative test results for the given population. Within the T1a-high risk population, a reduction rate of 68.5% was achieved with an NPV of 97.5%. Similarly, in the T1b population, there was a reduction rate of 40.9% with an NPV of 97.8%. Moreover, by ruling out patients with a <5% risk, the i31-GEP increased the overall yield of eligible T1 and T2 patients by 3% over positivity rates as calculated only with clinicopathologic factors (Table 6).

i31-GEP Survival Outcomes

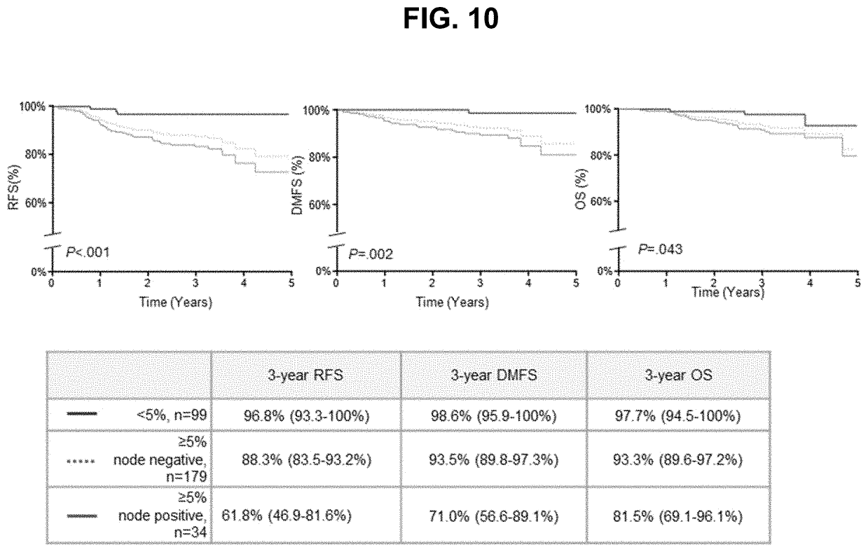

The study included cases from a prospective, multi-center U.S. study that was recently published that had data on SLN status and 3.2 years median follow-up, allowing for assessment of patient outcomes in the <5% and >5% risk group described by the i31-GEP model. Patients predicted by the i31-GEP to have <5% SLN positivity risk had significantly higher RFS (96.8% [95% CI 93.3-100%] vs. 88.3% [95% CI 83.5-93.2%] than patients predicted to have ≥5% risk and were node-negative and vs. 61.8% [95% CI 46.9-81.6%] than patients predicted to have ≥5% risk and were node-positive, P<0.001]), DMFS 98.6% [95% CI 95.9-100%) vs. 93.5% [95% CI 89.8-97.3%] than patients predicted to have ≥5% risk and were node-negative and vs. 71.0% [95% CI 56.6-89.1%] than patients predicted to have ≥5% risk and were node-positive, P=0.002]), and OS (97.7% [95 CI 94.5-100%] vs. 93.3% [95% CI 89.6-97.2%]) than patients predicted to have ≥5% risk and were node-negative and vs. 81.5% [95% CI 69.1-96.1%] than patients predicted to have ≥5% risk and were node-positive, P=0.043]) (FIG. 10). These data support current national guidelines that patients with <5% risk, in this case as identified by the i31-GEP model, would be expected to have high survival rates and are unlikely to experience harm from foregoing an SLNB. As expected, a positive SLNB in the >5% risk group negatively affected overall outcomes.

### Discussion:

While NCCN guidelines recommend SLNB in patients with >10% SLN positivity risk, 88% of patients who undergo an SLNB receive a negative result, risk unnecessary adverse events resulting from surgical intervention, and retain their initially diagnosed AJCC stage. Better identification of patients who can safely forego SLNB would have a major impact on surgery-associated morbidity and healthcare costs; and conversely those identified as having a higher likelihood of SLN positivity and a concomitant higher rate of metastasis would benefit from increased healthcare resource allocation. This disclosure demonstrates that integration of clinicopathologic features with the continuous 31-GEP score, determined from primary tumor tissue, improves the identification of patients with SLN metastasis risks below the threshold of 5% established by the NCCN for recommending that the SLNB procedure not be performed, and identify patients with >10% risk for whom SLNB should be offered.

This study demonstrated that i31-GEP accurately identified a larger percentage of patients (27.7%, 464/1674) with a <5% risk of SLN positivity than were identified by T stage in conjunction with clinicopathologic risk factors without the 31-GEP (T1a-LR, 8.5%, 142/1674, Table 5). With increasing numbers of tumors being diagnosed in early stages, the misclassification of low-risk T1 tumors as high risk by the current standards may partially explain the high rate of negative SLNB results seen in T1 tumors in clinical practice. A recent nomogram by Lo et al. found 12.4% of patients with <5% SLN positivity risk (“Improved Risk Prediction Calculator for Sentinel Node Positivity in Patients With Melanoma: The Melanoma Institute Australia Nomogram.” J Clin Oncol. 2020 Jun. 12; JCO.19.02362). They further predicted that only 27% of patients with T1 tumors had a <5% risk compared with the i31-GEP that found 57.6% of T1 cases with <5% risk. On the other hand, some SLN prediction models have focused on higher risk populations. Bellomo et al. (“Model Combining Tumor Molecular and Clinicopathologic Risk Factors Predicts Sentinel Lymph Node Metastasis in Primary Cutaneous Melanoma.” JCO Precis Oncol. 2020 April; (4):319-34) analyzed a melanoma cohort where just 25% of patients have T1 tumors, all of which were T1b tumors, leaving 75% of their cohort with T2-T3 tumors. Further, the T1b tumors in their cohort were less risky as a group (<5% risk overall) than T1b tumors reported by NCCN (5-10% risk). Finally, Bellomo et al. use an unknown cut-off for high and low-risk patients and are moving away from personalized risk prediction. In contrast to Bellomo et al., 46% of the validation cohort in our study have T1 tumors with an even split between T1a (377) and T1b tumors (328), and the T1b SLN positivity rate of 6.5% (18/279) is in line with current guidelines. Further, a detailed analysis of T1a tumors with other high-risk features is provided, which can help clinicians determine who should consider an SLNB in this traditionally low-risk population, and is highlighted by the fact that in this study, nearly 5% of the T1a population with high-risk features were identified as having a >10% risk of SLN positivity. These data demonstrate that the i31-GEP offers a more personalized risk prediction for patients at low and high risk of SLN metastasis than overall T-stage alone, particularly for patients with T1 tumors. Of high clinical treatment plan importance, patients with <5% SLN positivity risk as predicted by the i31-GEP (FIG. 10) have high RFS, DMFS, and OS.

Given that many studies associate SLN positivity with clinicopathologic risk factors, the strength of the i31-GEP is that, in addition to the tumor biology as detected through the 31-GEP score, it incorporates routinely recorded clinicopathologic features, including Breslow thickness, MR, ulceration, and age to improve SLN positivity prediction. Notably, the continuous 31-GEP score is the most important feature in the algorithm and adds significant value to current guidelines by identifying both a larger number of patients with <5% SLN positivity risk than using clinicopathologic features alone as well as those with >10% risk. These data support integrating clinicopathologic features with the continuous 31-GEP score to improve the identification of patients most likely to benefit from either foregoing or receiving an SLNB. Importantly, the i31-GEP aligns with published data that SLN positivity risk is negatively associated with increasing age even though older patients have increased risk of death from CM, that SLN risk is positively associated with increasing Breslow thickness, mitotic rate, and presence of ulceration, and that patients with a low 31-GEP score (0-0.41) with advanced age have <5% risk of SLN positivity.

The NCCN guidelines recommend that patients with T1a tumors with high-risk features such as uncertain microstaging, lymphovascular invasion, or mitotic rate ≥2/mm2, particularly in those younger than 40, have a 5-10% risk of SLN positivity and should consider SLNB.(4) A low SLN positivity risk is confirmed in the validation cohort, in which no patient with a T1a tumor and no documented high-risk feature (0%, 0/30) who had the SLNB procedure performed had a positive SLN compared with 7.5% (7/93) with at least one high-risk feature (Table 8). Moreover, the i31-GEP improves SLNB guidance for patients with T1a tumors with high-risk features or T1b tumors predicted to have a 5%-10% SLN positivity risk. The i31-GEP re-classified 63% of patients from the 5-10% SLN positivity risk range to either <5% or >10% risk compared with T-stage-based risk predictions with or without high-risk clinicopathologic features. These data show that patient risk reclassification by incorporating clinicopathologic features with molecular tumor biology as assessed by the 31-GEP test can help guide discussions on whether a patient should forego or undergo an SLNB, respectively.

Consider a typical 60-year-old patient with a 0.5 mm tumor with no ulceration or regression and two mitoses/mm2. Current guidelines suggest that this patient's melanoma, classified as T1a with a high-risk feature, has between a 5% and 10% risk of a positive SLN, and an SLNB should be discussed with the patient and considered. However, incorporating the continuous 31-GEP score with clinicopathologic features gives a more precise risk estimate that could affect decision making. If the patient received a low risk (0-0.41; Class 1A) 31-GEP score (e.g., 0.0, the lowest score, their SLN positivity risk prediction by the i31-GEP would be 2.7%, which is under the 5% threshold provided by NCCN guidelines for considering an SLNB. However, if the patient received a high-risk (0.59-1.0, Class 2B) 31-GEP score (e.g., 0.73, the median Class 2B score, the risk of a positive SLN increases to 13.9%, above the 10% threshold at which NCCN guidelines recommend offering SLNB. This example shows the precision of the i31-GEP to identify patients at low or high risk of SLN positivity and exemplifies the additional layer of precision added by the 31-GEP to determine individualized, risk-aligned patient management strategies.

While the i31-GEP developed in this report was independently validated to refine risk assessment within the context of clinical, histological, and molecular features, there are some limitations. The populations from both the training and validation cohorts were mostly assessed at surgical oncology centers, with nearly 80% having an SLNB performed, and therefore may miss patients not referred out of a dermatology clinic. Additionally, while not obvious on pathology report review, there are some T1a patients that were evaluated clinically but did not have SLNB performed; therefore, it cannot be ruled out the potential for occult nodal metastases in the remaining patients who were clinically observed for nodal positivity at the time of diagnosis. In addition, data for TILs was confounding due to non-standard reporting criteria. The result of this variability is that TILs did not contribute to the model; future studies could determine if TILs is an important variable for SLNB decision making.

These data demonstrate the value of advanced artificial intelligence tools for personalized risk assessment, and the contribution of clinicopathologic features to the 31-GEP facilitates the precision necessary for patient management. By incorporating the 31-GEP with impactful clinicopathologic features into SLNB clinical decision making, the i31-GEP unlocks the potential to reduce the uncertainty of broad SLNB risk groups defined by the AJCC T-stage to more accurately identify patients whose true risk is below 5% or greater than 10%. The AJCC provides a generalized risk prediction that is limited to the mean population risk. The i31-GEP approach enables clinicians and patients to access a more refined risk prediction to guide patient management.

TABLE 4
Demographics and clinical characteristics of the training and validation cohort

| | Training Cohort | | Validation Cohort | | |
|---|---|---|---|---|---|
| | N = 1,398 | | N = 1,674 | | P-value |
| 31-GEP,* a.u. (range) | 0.35 | (0.00-1.00) | 0.40 | (0.00-1.00) | <.001 |
| Breslow thickness*, | 1.2 | (0.1-60.0) | 1.2 | (0.1-68.0) | .292^{#} |
| mm (range) | | | | | |
| Ulceration present, | 21.6% | (302/1398) | 23.6% | (395/1674) | .195^{§} |
| % (n/N) | | | | | |
| Mitotic Rate*, 1/mm² (range) | 1.0 | (0-74.0) | 1.0 | (0-235.0) | <.001^{#} |
| Absence of TILs, % (n/N) | 13.3% | (186/1398) | 9.9% | (165/1674) | .003^{§} |
| Presence of Microsatellites, | 0.4% | (5/1398) | 1.1% | (18/1674) | .022^{§} |
| % (n/N) | | | | | |
| Transected base, % (n/N) | 19.5% | (272/1398) | 34.9% | (585/1674) | <.001^{§} |
| Presence of Regression, | 13.7% | (191/1398) | 14.6% | (245/1674) | .437^{§} |
| % (n/N) | | | | | |
| Lymphovascular Invasion, | 2.8% | (39/1398) | 3.2% | (54/1674) | .526^{§} |
| % (n/N) | | | | | |
| Histologic subtype | | | | | |
| Superficial spreading | 368/1398 | (26.3%) | 512/1674 | (30.6%) | .010^{§} |
| Nodular | 167/1398 | (11.9%) | 304/1674 | (18.2) | |
| Other/Unspecified | 863/1398 | (61.7%) | 858/1674 | (51.2%) | |
| Tumor location | | | | | |
| Head and neck | 282/1398 | (20.2%) | 352/1674 | (21.0%) | .591^{§} |
| Trunk | 559/1398 | (40.0%) | 679/1674 | (40.6%) | |
| Extremity | 549/1398 | (39.3%) | 638/1674 | (31.8%) | |
| Sex, % male (n/N) | 54.7% | (765/1398) | 55.1% | (923/1674) | .537^{§} |
| Age*, years (range) | 62.3 | (18.0-95.4) | 65.2 | (20.6-96.6) | <.001^{#} |
| Total SLN positive, | 10.4% | (145/1398) | 11.1% | (186/1674) | .521^{§} |
| % (n/N) | | | | | |
| SLNB Performed, % (n/N) | 79.5% | (1111/1398) | 75.1% | (1258/1674) | .003^{§} |
| SLNB positive, % (n/N) | 12.9% | (143/1111) | 14.2% | (179/1258) | .368^{§} |

*Median continuous value;^{#}Mann-Whitney U test;^{§}Fisher’s exact test

TABLE 5
The i31-GEP improves the precision of T-stage predicted SLN positivity risk estimates

| | Standard system of risk binning*; | | | Precision risk reclassification | | | | | | | |
|---|---|---|---|---|---|---|---|---|---|---|---|
| | % population (n) | | | by i31-GEP, % population (n) | | | | | | | |
| | Not | | | Not | | | | | | | |
| T stage | recommended | Considered | Recommended | recommended | | Considered | | Recommended | | Percent | |
| (n) | (<5%) | (5-10%) | (>10%) | (<5%) | | (5-10%) | | (>10%) | | Change ** | |
| T1a-LR | 100%(142) | — | — | 78.2% | (111) | 21.1% | (30) | 0.7% | (1) | 21.8% | (31) |
| (142) | | | | | | | | | | | |
| T1a-HR | — | 100%(235) | — | 68.5% | (161) | 26.8% | (63) | 4.7% | (11) | 73.2% | (172) |
| (235) | | | | | | | | | | | |
| T1b | — | 100%(328) | — | 40.9% | (134) | 44.8% | (147) | 14.3% | (47) | 55.2% | (181) |
| (328) | | | | | | | | | | | |
| T2a | — | — | 100%(416) | 12.5% | (52) | 44.7% | (186) | 42.8% | (178) | 57.2% | (238) |
| (416) | | | | | | | | | | | |
| T2b | — | — | 100%(118) | 4.2% | (5) | 44.1% | (52) | 51.7% | (61) | 48.3% | (57) |
| (118) | | | | | | | | | | | |
| T3a | — | — | 100%(164) | 0% | (0) | 14.6% | (24) | 85.4% | (140) | 14.6% | (24) |
| (164) | | | | | | | | | | | |
| T3b | — | — | 100%(139) | 0.7% | (1) | 5.0% | (7) | 94.2% | (131) | 5.8% | (8) |
| (139) | | | | | | | | | | | |
| T4a | — | — | 100%(51) | 0% | (0) | 7.8% | (4) | 92.2% | (47) | 7.8% | (4) |
| (51) | | | | | | | | | | | |
| T4b | — | — | 100%(81) | 0% | (0) | 1.2% | (1) | 98.8% | (80) | 1.2% | (1) |
| (81) | | | | | | | | | | | |

*Classification of risk according to the NCCN guidelines by T- stage.
** Percent changed from risk bin designated by T stage
T1a-LR (low-risk): T1a with no recorded high-risk features; T1a-HR (high-risk): T1a with one or more feature that may be considered high risk when assessing SLNB eligibility including age <40 yrs, mitotic rate ≥2/mm2, presence of regression, lymphovascular invasion, transected base, or absence of TILs.

TABLE 6
Accuracy of the i31-GEP by T-stage

| | T1aHR- | | | | |
|---|---|---|---|---|---|
| | T2 | T1a HR | T1b | T2a | T2b |
| Negative | 97.4% | 97.5% | 97.8% | 96.2% | 100.0% |
| predictive value | | | | | |
| False-negative | 2.6% | 2.5% | 2.2% | 3.8% | 0.0% |
| rate | | | | | |
| Reduction rate | 32.1% | 68.5% | 40.9% | 12.5% | 4.2% |
| Sensitivity | 89.8% | 42.9% | 83.3% | 95.8% | 100.0% |
| Pre-test SLN | 8.0% | 3.0% | 5.5% | 11.5% | 12.7% |
| positivity rate | | | | | |
| Yield (PPV) | 10.6% | 4.1% | 7.7% | 12.6% | 13.3% |

<5.0% risk of SLN positivity was considered a negative test result, and ≥5% risk of SLN positivity was considered a positive test result. Accuracy was assessed using all T1a-HR (high risk), T1b, T2a, and T2b cases. There were no positive SLNs in the T1a group with no high-risk features. Conversely, there were no negative i31-GEP test results in the T3a, T4a and T4b populations and only 1 negative test result (in a patient with a negative SLN) in the T3b population. Therefore, they were excluded from analysis.

TABLE 7
Variable importance in SLN positivity prediction.

| | Variable | Log- | |
|---|---|---|---|
| | importance | likelihood | |
| | assessment | value | Spearman |
| | function* | (G²)** | Correlation |
| 31-GEP score | 100 | G² = 91.3; | r = 0.24; |
| (continuous) | | P < .001 | P < .001 |
| Mitotic rate | 46 | G² = 20.7; | r = 0.14; |
| (continuous) | | P < .001 | P < .001 |
| Breslow’s thickness | 37 | G² = 53.5; | r = 0.25; |
| (continuous) | | P < .001 | P < .001 |
| Ulceration | 21 | G² = 19.1; | r = 0.12; |
| (categorical) | | P < .001 | P < .001 |
| Age | 21 | G² = 10.5; | r = −0.09; |
| (continuous) | | P = .001 | P = .001 |

* Scale of 0-100 with 100 having the highest importance.
**Highest G² value corresponds to the best explanatory variable

TABLE 8
Pre-test SLN positivity rates by T-stage
in 1674 patients with T1-T4 CM

| | SLNB assessed | | |
|---|---|---|---|
| | | % SLN | |
| T-stage | n/N | Positive | 95% CI |
| T1-T4 | 180/1258 | 14.3% | 12.4-16.4% |
| T1a-LR | 0/30 | 0% | 0-11.6% |
| T1a-HR | 7/93 | 7.5% | 3.1-14.9% |
| T1b | 18/279 | 6.5% | 3.9-10.0% |
| T2a | 48/378 | 12.7% | 9.5-16.5% |
| T2b | 15/106 | 14.2% | 8.1-22.3% |
| T3a | 32/147 | 21.8% | 15.4-29.3% |
| T3b | 30/119 | 25.2% | 17.7-34.0% |
| T4a | 8/42 | 19.0% | 8.6-34.1% |
| T4b | 22/64 | 34.4% | 23.0-47.3% |

T1a-LR: T1a with no documented high-risk feature;
T1a-HR, T1a with one or more high risk clinicopathologic features

### IV. Conclusion

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims.

The above detailed description describes various features and functions of the disclosed systems, devices, and methods with reference to the accompanying figures. In the figures, similar symbols typically identify similar components, unless context dictates otherwise. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

With respect to any or all of the message flow diagrams, scenarios, and flow charts in the figures and as discussed herein, each step, block, operation, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step, block, or operation that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer-readable medium such as a storage device including RAM, a disk drive, a solid state drive, or another storage medium.

Moreover, a step, block, or operation that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims.

What is claimed is:

1. A non-transitory, computer-readable medium having instructions stored thereon, wherein the instructions, when executed by a processor, cause the processor to execute a method comprising:
obtaining a plurality of clinical-pathologic factors related to a patient, wherein the clinical-pathologic factors are indicative of risk associated with melanoma;
obtaining a continuous multigene-expression profile score for the patient, wherein the continuous multigene-expression profile score is based on multiple genes whose expressions are related to melanoma;
determining, based on the plurality of clinical-pathologic factors and the continuous multigene-expression profile score, a risk score for the patient; and
outputting the risk score for use in determining a prognosis and treatment plan.

2. The non-transitory, computer-readable medium of claim 1, wherein obtaining the continuous multigene-expression profile score comprises:
receiving a continuous multigene-expression profile for the patient based on multiple genes whose expressions are related to melanoma; and
calculating the continuous multigene-expression profile score based on the continuous multigene-expression profile.

3. The non-transitory, computer-readable medium of claim 1, wherein the continuous multigene-expression profile score comprises a score between 0 and 1 that represents expressions of 31 different genes relating to melanoma.

4. The non-transitory, computer-readable medium of claim 1, wherein the method further comprises:
obtaining, after outputting the risk score, one or more additional clinical-pathologic factors related to the patient;
calculating, based on the plurality of clinical-pathologic factors, the one or more additional clinical-pathologic factors, and the continuous multigene-expression profile score, a revised risk score for the patient; and
outputting the revised risk score for use in determining a prognosis and treatment plan.

5. The non-transitory, computer-readable medium of claim 1, wherein outputting the risk score comprises:
generating a clinical laboratory report usable for patient care; and
causing an associated printing device to print the clinical laboratory report.

6. The non-transitory, computer-readable medium of claim 1, wherein the plurality of clinical-pathologic factors comprises an age of the patient, a gender of the patient, a tumor site location, a histologic type, a Breslow thickness measurement, a transected base measurement, an ulceration measurement, a microsatellites measurement, a mitotic rate, a lymphovascular invasion measurement, a tumor infiltrating lymphocytes measurement, a tumor regression, a sentinel lymph node status, or an in-transit disease/satellites measurement.

7. The non-transitory, computer-readable medium of claim 1, wherein the risk score comprises a sentinel lymph node (SLN) metastasis positivity, a recurrence-free survival (RFS) rate, a distance metastasis-free survival (DMFS) rate, or a melanoma specific survival (MSS) rate.

8. The non-transitory, computer-readable medium of claim 1, wherein the method further comprises:
receiving user login credentials; and
validating the user login credentials by comparing the user login credentials to stored credentials associated with a plurality of authenticated users.

9. The non-transitory, computer-readable medium of claim 8, wherein the plurality of authenticated users comprises physicians or clinicians permitted to provide and access information associated with the patient.

10. The non-transitory, computer-readable medium of claim 8, wherein the plurality of authenticated users comprises the patient.

11. The non-transitory, computer-readable medium of claim 1, wherein outputting the risk score comprises providing the risk score to an electronic health record associated with the patient.

12. The non-transitory, computer-readable medium of claim 1,
wherein the plurality of clinical-pathologic factors are received from user input into a browser-based application,
wherein the continuous multigene-expression profile score for the patient is received from user input into the browser-based application, and
wherein outputting the risk score comprises displaying the risk score via the browser-based application.

13. The non-transitory, computer-readable medium of claim 1,
wherein the plurality of clinical-pathologic factors are received from user input into a mobile application,
wherein the continuous multigene-expression profile score for the patient is received from user input into the mobile application, and
wherein outputting the risk score comprises causing an associated user interface to display the risk score via the mobile application.

14. The non-transitory, computer-readable medium of claim 1, wherein the method further comprises:
determining, based on the plurality of clinical-pathologic factors, a range of risk scores for use in determining a prognosis and treatment plan; and
outputting the range of risk scores.

15. The non-transitory, computer-readable medium of claim 1, wherein determining the risk score for the patient comprises applying a machine-learned model to the plurality of clinical-pathologic factors and the continuous multigene-expression profile score.

16. The non-transitory, computer-readable medium of claim 15, wherein the machine-learned model comprises an artificial neural network, and wherein applying the machine-learned model to the plurality of clinical-pathologic factors comprises applying machine-learned weights of the artificial neural network to each of the clinical-pathologic factors and the continuous multigene-expression profile score.

17. The non-transitory, computer-readable medium of claim 1, wherein determining the risk score for the patient comprises applying a statistical model to the plurality of clinical-pathologic factors and the continuous multigene-expression profile score.

18. A method comprising:
determining a plurality of clinical-pathologic factors related to a patient, wherein the clinical-pathologic factors are indicative of risk associated with melanoma;
determining a continuous multigene-expression profile score for the patient, wherein the continuous multigene-expression profile score is based on multiple genes whose expressions are related to melanoma;
providing the plurality of clinical-pathologic factors and the continuous multigene-expression profile score to a computing device, wherein the computing device is configured to:
calculate, based on the plurality of clinical-pathologic factors and the continuous multigene-expression profile score, a risk score for the patient; and
output the risk score; and
modifying a prognosis or treatment plan based on the risk score.

19. The method of claim 18, wherein modifying the prognosis or treatment plan based on the risk score comprises determining that further diagnostic testing is to be performed or performing further diagnostic testing.

20. The method of claim 18, wherein modifying the prognosis or treatment plan based on the risk score comprises performing a sentinel lymph node (SLN) biopsy on the patient, wherein the method further comprises providing results from the SLN biopsy on the patient to the computing device, and wherein the computing device is further configured to:
calculate, based on the plurality of clinical-pathologic factors, the results from the SLN biopsy, and the continuous multigene-expression profile score, a revised risk score for the patient; and
output the revised risk score.

21. The method of claim 18, wherein determining the continuous multigene-expression profile score comprises providing a continuous multigene-expression profile to the computing device, and wherein the computing device is further configured to calculate the continuous multigene-expression profile score based on the continuous multigene-expression profile.

22. The method of claim 18, wherein determining the plurality of clinical-pathologic factors or determining the continuous multigene-expression profile score comprises:
performing one or more laboratory tests using one or more samples from the patient;
receiving demographic information from the patient; or
accessing one or more records associated with the patient.
